SEQUENCE LISTING (15 sequences)

SEQ ID NO: 1 - protein, 18 aa, Artificial Sequence
  alpha-isoform survivin S1 positions 17-34
HRISTFKNWPFLEGCACT

SEQ ID NO: 2 - protein, 27 aa, Artificial Sequence
  alpha-isoform survivin S2 positions 84-110
CAFLSVKKQFEELTLGEFLKLDRERAK

SEQ ID NO: 3 - protein, 21 aa, Artificial Sequence
  alpha-isoform survivin S3 positions 122-142
KEFEETAKKVRRAIEQLAAMD

SEQ ID NO: 4 - protein, 15 aa, Artificial Sequence
  alpha-isoform survivin S1 positions 17-31
HRISTFKNWPFLEGC

SEQ ID NO: 5 - protein, 15 aa, Artificial Sequence
  alpha-isoform survivin S1 positions 20-34
STFKNWPFLEGCACT

SEQ ID NO: 6 - protein, 15 aa, Artificial Sequence
  alpha-isoform survivin S2 positions 84-98
CAFLSVKKQFEELTL

SEQ ID NO: 7 - protein, 15 aa, Artificial Sequence
  alpha-isoform survivin S2 positions 90-104
KKQFEELTLGEFLKL

SEQ ID NO: 8 - protein, 15 aa, Artificial Sequence
  alpha-isoform survivin S2 positions 93-107
FEELTLGEFLKLDRE

SEQ ID NO: 9 - protein, 15 aa, Artificial Sequence
  alpha-isoform survivin S2 positions 96-110
LTLGEFLKLDRERAK

SEQ ID NO: 10 - protein, 15 aa, Artificial Sequence
  alpha-isoform survivin S3 positions 122-136
KEFEETAKKVRRAIE

SEQ ID NO: 11 - protein, 15 aa, Artificial Sequence
  alpha-isoform survivin S3 positions 128-142
AKKVRRAIEQLAAMD

SEQ ID NO: 12 - DNA, 26 bp, Artificial Sequence
  Chemically synthesized oligonucleotide - Litenimod
taaacgttataacgttatgacgtcat

SEQ ID NO: 13 - DNA, 24 bp, Artificial Sequence
  Chemically synthesised oligonucleotide - CpG 2006
tcgtcgttttgtcgttttgtcgtt

SEQ ID NO: 14 - DNA, 21 bp, Artificial Sequence
  Chemically synthesized oligonucleotide - CpG 2336
ggggacgacgtcgtggggggg

SEQ ID NO: 15 - protein, 142 aa, Homo sapiens
  Alpha-isolform Survivin
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

(19) United States
(12) Patent Application Publication
(10) Pub. No.: US 2020/0061173 A1
(43) Pub. Date: Feb. 27, 2020

(54) IMMUNOGENIC COMPOSITION COMPRISING SURVIVIN PEPTIDES

(71) Applicant: Vaxeal Research SAS, Evry (FR)

(72) Inventors: Ahmed Bouzidi, Lausanne (CH); Jérôme Kerzerho, Palaiseau (FR)

(73) Assignee: Vaxeal Research SAS, Evry (FR)

(21) Appl. No.: 16/087,381

(22) PCT Filed: Mar. 15, 2017
(86) PCT No.: PCT/EP2017/056159

§ 371 Date: Sep. 21, 2018

(30) Foreign Application Priority Data
Mar. 21, 2016 (GB) 1604755.7

(51) Int. Cl. A61K 39/00; A61K 39/39; A61P 35/00; A61P 37/04

(52) U.S. Cl. CPC A61K 39/00115; A61K 39/39; A61P 35/00; A61P 37/04; A61K 2039/55516; A61K 2039/572; A61K 2039/55566; A61K 2039/55572; A61K 2039/70; A61K 2039/55561; A61K 2039/55522

(57) ABSTRACT

The present invention relates to immunogenic compositions, in particular, immunogenic compositions comprising at least one peptide derived from survivin, or a functional derivative thereof. Uses of the immunogenic compositions in the treatment of cancer, in particular a cancer over-expressing survivin are also disclosed.

The present invention relates to immunogenic compositions, in particular, immunogenic compositions comprising at least one peptide derived from survivin, or a functional derivative thereof. Uses of the immunogenic compositions in the treatment of cancer, in particular a cancer over-expressing survivin are also disclosed.

### BACKGROUND

Despite recent progress in surgical and standard cancer therapy approaches, several cancers are still difficult to treat and cure, especially in patients with advanced stages of the disease. Therefore, new therapeutic strategies are needed and immunotherapeutic approaches targeting tumour-associated antigens (TAAs) are among the most prominent approaches recently developed. Immunotherapeutic cancer vaccines have received attention due to their specificity and minimal damage when compared to conventional cancer therapies such as chemotherapy and radiotherapy.

One aim of cancer therapies is the induction of effective anti-tumour immunity in cancer patients leading to elimination of tumours and memory responses for long-lasting protection against relapses. Modern therapeutic vaccination has been shown to elicit tumour antigen specific T-cell immunity. However, conventional cancer vaccines are showing modest clinical effects such as objective tumour responses in only a small proportion of immunized patients and increase in overall survival for only a few vaccines.

Studies have identified several hurdles that can limit efficacy of conventional cancer vaccines. For example, these can arise from the targeting of inappropriate tumour antigens as some cancer vaccines e.g. targeting non-essential antigens, leading to their weak immunogenicity as malignant cells can evade immunological surveillance by decreasing or repressing the expression of these antigens. Some other vaccines have a restricted efficacy to certain cancer indications, as they do not target broadly expressed tumour antigens.

Some other hurdles arise from the sub-optimal composition and design of the tumour antigen-based vaccines. This can lead to weak immunogenicity, restricted efficacy to certain cancer indications (e.g. not use of T cell epitopes covering a large array of HLA molecules) and difficulty in generating a robust memory response, and in achieving the right balance of CD4⁺ and CD8⁺ T-cells.

For a long time, CD8⁺ T cells or cytotoxic T lymphocytes (CTLs) have been considered to be the main protagonists among adaptive immune cells involved in antitumor responses, predominantly because they exhibit cytotoxic activity towards tumour cells expressing TAAs. However, it has been shown that CD4⁺ T helper 1 (Th1) lymphocytes also play a key role in orchestrating the antitumor immune response. Once activated, Th1 cells which are mainly characterized by INF-γ production, contribute to the induction and maintenance of cytotoxic response mediated by CD8⁺ T cells against tumours and notably inducing the activation of dendritic cells through cell contacts and the secretion of numerous cytokines (Shedlock and Shen 2003; Church et al. 2014; Sharma et al. 2013; Ostrand-Rosenberg 2005).

However, CD4⁺ T lymphocytes can mediate direct antitumor effect, even in the absence of CD8⁺ T cells. They can also exert indirect antitumor activity via CTL-independent mechanisms, by recruiting and activating innate immune cells such as natural killers and macrophages but also through targeting of tumour stroma and inhibition of angiogenesis (H. Kim and Cantor 2014; Haabeth et al. 2014).

Several studies have demonstrated that immunotherapeutic vaccines activating both CTLs and CD4⁺ T cells are more effective to induce tumour regression. Short peptide- and DNA-based vaccines mainly induced CD8⁺ T cells while recombinant proteins mainly induced CD4⁺ T cells.

Survivin (16.5 kDa) is the smallest member of a family of apoptosis inhibitors (IAPs). It is encoded by a complex gene called BIRC5 (Baculoviral IAP repeat-containing protein 5), located on human chromosome 17 (17q25) and containing four well defined and three hidden exons. Alternative splicing of pre-mRNA generates five splice variants namely Survivin wild type (wt, alpha isoform), Survivin-2α, Survivin-2B, Survivin-Δ3Ex, Survivin-3B, and Survivin-3α.

Survivin wt comprises exons 1, 2, 3 and 4 and encode the alpha-isoform of survivin (SEQ ID NO: 15, 142 amino acids, GenBank AAC51660 or SwissProt 015392). Survivin-2B is characterized by introducing a new exon of 69 bp with pro-apoptotic activity. Survivin-Ex3 has exon 3 excluded, and like the wild type, carries anti-apoptotic activity. Survivin-3B has inclusion of a part of intron 3, preserving a complete BIR domain with anti-apoptotic activity. Survivin-2α, the smallest survivin transcript, includes a 197 bp region of the 3′ end of intron 2, resulting in a truncated version of the BIR domain also having pro-apoptotic function. Protein and mRNA levels of the pro- and anti-apoptotic isoforms of survivin have been found to be associated with aggressive phenotypes of cancers (Boidot, Vegran, and Lizard-Nacol 2009).

Survivin is almost undetectable in most healthy/normal adult tissues, and its expression is largely limited to developing embryos but also CD34⁺ haematopoietic stem cells, and epithelial and gonadal cell lines.

In contrast, survivin is overexpressed in nearly all human cancers, including breast, liver, colon, lung, ovarian, uterine, oesophageal, stomach, pancreatic and prostate cancers, but also Hodgkin's disease, and melanomas, non-Hodgkin's lymphomas, leukemias, neuroblastomas, pheochromocytomas, soft tissue sarcomas and brain tumours (Andersen et al. 2007; Khan et al. 2015; Adamkov et al. 2012; Ahmed et al. 2012; Waligórska-Stachura et al. 2012; Xie et al. 2013; M. Zhang et al. 2009; Baytekin et al. 2011).

Survivin has essential roles in inhibiting apoptosis, by repressing the activity of caspases 3, 7 and 9, and in controlling proper cell division by notably participating in the formation of the CPP (chromosomal passenger protein) complex which comprises the Aurora B kinase, the 1NCENP protein (inner centromere protein) and TD60 (Telophase disk antigen) (Fortugno et al. 2002; Li et al. 1998).

Accordingly, in tumour cells, the overexpression of survivin leads to an insensitivity to apoptosis and promotes cell division. In other words, cells do not die as a consequence of apoptotic triggers, but rather keep on proliferating.

However, survivin is also implicated in the control of diverse other cellular functions, including surveillance checkpoints, suppression of cell death, the regulation of mitosis, and adaptation to unfavourable environments (Altieri 2003; Altieri 2006). As a result, survivin-expressing cancers correlate with poor prognosis for the patients (Adida et al. 2000; Adamkov et al. 2012; Ahmed et al. 2012; Waligórska-Stachura et al. 2012; Xie et al. 2013; M. Zhang et al. 2009; Baytekin et al. 2011). On the other hand, inhibition of the expression of survivin by antisense oligonucleotides induced tumour apoptosis and increased tumour sensitivity to chemotherapy, demonstrating that survivin is essential to the survival of tumour cells (Olie et al. 2000; D. Yang, Welm, and Bishop 2004; Altieri 2003; Gao et al. 2015; Minoda et al. 2015; Groner and Weiss 2014).

Studies into the spontaneous CD4⁺ and CD8⁺ T cell responses directed against Survivin, in various cancer patients, indicates that this tumor antigen can induce such cellular responses and demonstrates the absence of T cell tolerance against this tumor antigen in tumor bearing patients (Wang et al. 2008; Piesche et al. 2007; Schaue et al. 2008; Andersen et al. 2001; Turksma et al. 2013; Casati et al. 2003; Karanikas et al. 2009).

Survivin-specific antibody responses have also been detected in individuals suffering from lung, colorectal, breast, esophageal, gastric, pharyngeal cancers. However, no beneficial or detrimental outcome has been observed (Karanikas et al. 2009; Rohayem et al. 2000; Megliorino et al. 2005).

Survivin derived CD8⁺ T epitopes restricted to the human leucocyte antigen (HLA) A1, A2, A3, A11, A24, B7, B8, B15 and B35 molecules of the major histocompatibility complex class I (HLA I molecules) have been identified (S Reker et al. 2004; Andersen et al. 2001, Schmitz et al. 2000; Siegel et al. 2004; Wang et al. 2008; Andersen et al. 2006; Hirohashi et al. 2002; Ohtake et al. 2014; Sine Reker et al. 2004; WO 2007/039192, WO 2006/081826, and WO 2004/067023). Several Survivin derived CD4⁺ T cell epitopes restricted to various HLA-DR and HLA-DP4 molecules of the major histocompatibility complex class II (HLA II molecules) have also been identified including promiscuous CD4⁺ T cell epitopes presenting several HLA II molecules (Tanaka et al. 2011; Kim et al. 2008; Piesche et al. 2007; Wang et al. 2008; WO 2007/036638 and WO 2009/123188).

It has also been proposed to use survivin (recombinant protein), an expression vector for this antigen, short CD4⁺ or CD8⁺ T cell epitopes derived from this antigen or dendritic cells transfected with such an expression vector or loaded with such T cell epitopes, as an antitumor vaccine (WO 2000/03693, WO 2006/081826, WO 2009/012460, and WO 2007/039192).

However, despite the relevance of survivin as a target for antitumor immunization, the clinical effects of these cancer vaccines have been limited and attempts in the prior art to provide viable vaccine candidates targeting survivin capable of inducing effective anti-tumoral T cell responses in the majority of the immunized patients, have been largely unsuccessful. Therefore, there is a need for identifying and developing further approaches that target cancers over-expressing survivin in order to develop an effective and successful cancer therapy.

### SUMMARY

According to a first aspect of the present invention, there is provided an immunogenic composition comprising:
(a) at least one peptide derived from the alpha-isoform of survivin, or functional derivative thereof;
(b) at least one immunostimulatory adjuvant; and
(c) at least one adjuvant capable of creating a depot effect.

The at least one adjuvant capable of creating a depot effect in (c) may comprise one or more adjuvants selected from the group consisting: alum, emulsion based formulations, mineral oil, non-mineral oil, and oil-in-water emulsion.

In some embodiments, the at least one adjuvant capable of creating depot effect in (c) comprises a Montanide® adjuvant.

The Montanide® adjuvant may be one selected from the group consisting: MR-59, ASO3, ISA-51 VG and ISA-720 VG (Seppic ISA series).

In some embodiments, the at least one immunostimulatory adjuvant in (b) may be an immunostimulatory oligonucleotide adjuvant comprising one or more unmethylated CpG motifs.

The immunostimulatory oligonucleotide adjuvant may comprise an oligodeoxynucleotide-containing unmethylated CpG motif (CpG-ODN).

In some embodiments, at least one immunostimulatory adjuvant in (b) may comprise a granulocyte macrophage colony-stimulating factor (GM-CSF) adjuvant.

In an alternative embodiment, (b) comprises an unmethylated CpG motif and a granulocyte macrophage colony-stimulating factor (GM-CSF).

For example, (b) may comprise an oligodeoxynucleotide-containing unmethylated CpG motif (CpG-ODN) and a granulocyte macrophage colony-stimulating factor (GM-CSF).

In a further alternative embodiment, (b) may comprise an unmethylated CpG motif and (c) may comprise a Montanide® adjuvant.

The unmethylated CpG motif may be an oligodeoxynucleotide-containing unmethylated CpG motif (CpG-ODN). The Montanide® adjuvant may be one selected from the group consisting: MR-59, ASO3, ISA-51 VG and ISA-720 VG (Seppic ISA series).

In some embodiments, the at least one peptide in (a) of the immunogenic composition comprises one or more peptides selected from the group consisting:
(i) a peptide of 15 to 18 consecutive amino acids located between positions 17 to 34 (SEQ ID NO: 1) of the alpha-isoform of survivin;
(ii) a peptide of 15 to 27 consecutive amino acids located between positions 84 to 110 (SEQ ID NO: 2) of the alpha-isoform of survivin; or
(iii) a peptide of 15 to 21 consecutive amino acids located between positions 122 to 142 (SEQ ID NO: 3) of the alpha-isoform of survivin.

In some embodiments, component (a) of the immunogenic composition may comprise one or more peptides selected from the group consisting:
(i) a peptide of 18 consecutive amino acids located between positions 17 to 34 (SEQ ID NO: 1) of the alpha-isoform of survivin;
(ii) a peptide of 27 consecutive amino acids located between positions 84 to 110 (SEQ ID NO: 2) of the alpha-isoform of survivin; or
(iii) a peptide of 21 consecutive amino acids located between positions 122 and 142 (SEQ ID NO: 3) of the alpha-isoform of survivin.

In some embodiments, the at least one peptide in (a) of the immunogenic composition may comprise one or more peptides selected from the group consisting:
(i) a peptide of 15 to 18 consecutive amino acids located between positions 17 to 34 (SEQ ID NO: 1) of the alpha-isoform of survivin, comprising at least the 15 consecutive amino acids between positions 20 to 34 (SEQ ID NO: 5), or positions 17 to 31 (SEQ ID NO: 4) of the alpha-isoform of survivin;
(ii) a peptide of 15 to 27 consecutive amino acids located between positions 84 to 110 (SEQ ID NO: 2) of the alpha-isoform of survivin, comprising at least the 15 consecutive amino acids between positions 84 to 98 (SEQ ID NO: 6), positions 90 to 104 (SEQ ID NO: 7), positions 93 to 107 (SEQ ID No: 8) or positions 96 to 110 (SEQ ID NO: 9) of the alpha-isoform of survivin; or
(iii) a peptide of 15 to 21 consecutive amino acids located between positions 122 to 142 (SEQ ID NO: 3) of the alpha-isoform of survivin, comprising at least the 15 consecutive amino acids between positions 122 to 136 (SEQ ID NO: 10) or 128 to 142 (SEQ ID NO: 11) of the alpha-isoform of survivin.

In the described embodiments, the sequence of the alpha-isoform of survivin is the sequence according to SEQ ID NO: 15.

In specific examples, the invention is directed to isolated variants of survivin proteins comprising, or in the alternative consisting of an amino acid sequence that is at least 80% identical to residues 17 to 34 (SEQ ID NO: 1), or an amino acid sequence that is at least 80% identical to residues 84 to 110 (SEQ ID NO: 2), or an amino acid sequence that is at least 80% identical to residues 122 to 142 (SEQ ID NO: 3), or an amino acid sequence that is at least 80% identical to residues 17 to 31 (SEQ ID NO: 4), or an amino acid sequence that is at least 80% identical to residues 20 to 34 (SEQ ID NO: 5), or an amino acid sequence that is at least 80% identical to residues 84 to 98 (SEQ ID NO: 6), or an amino acid sequence that is at least 80% identical to residues 90 to 104 (SEQ ID NO: 7), or an amino acid sequence that is at least 80% identical to residues 93 to 107 (SEQ ID No: 8), or an amino acid sequence that is at least 80% identical to residues 96 to 110 (SEQ ID NO: 9), or an amino acid sequence that is at least 80% identical to residues 122 to 136 (SEQ ID NO: 10), or an amino acid sequence that is at least 80% identical to residues 128 to 142 (SEQ ID NO: 11).

In additional embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 17 to 34 (SEQ ID NO: 1).

In more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 84 to 110 (SEQ ID NO: 2).

In still more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 122 to 142 (SEQ ID NO: 3).

In still more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 17 to 31 (SEQ ID NO: 4).

In still more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 20 to 34 (SEQ ID NO: 5).

In still more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 84 to 98 (SEQ ID NO: 6).

In still more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 90 to 104 (SEQ ID NO: 7).

In still more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 93 to 107 (SEQ ID No: 8).

In still more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 96 to 110 (SEQ ID NO: 9).

In still more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 122 to 136 (SEQ ID NO: 10).

In still more embodiments, the invention is directed to isolated survivin proteins that have amino acid sequences comprising, or in the alternative consisting of sequences, that are at least 85%, 86%, 87%, 88%, 89%, 90%, 91%, 92%, 93%, 94%, 95%, 96%, 97%, 98%, 99% and 100% identical to residues 128 to 142 (SEQ ID NO: 11).

In some embodiments, at least one peptide in (a) of the immunogenic composition is labelled or complexed. The peptide may be labelled or complexed, for example, with a tracking entity.

In some embodiments, (a) comprises a polypeptide. The polypeptide may comprise a concatenation of at least two peptides, wherein at least one of said concatenated peptides is a peptide as described herein according to the present invention.

In some embodiments, (a) comprises a lipopeptide. The lipopeptide may comprise a peptide or a polypeptide as described herein according to the present invention with a lipid added to an alpha-amino function or a reactive function of a side chain of said peptide or polypeptide.

In some embodiments, (a) comprises an expression vector. The expression vector may comprise a polynucleotide encoding a peptide, polypeptide or lipopeptide as described herein according to the present invention.

In an embodiment, the immunogenic composition may comprise:
(a) (i) a peptide of 18 consecutive amino acids located between positions 17 to 34 (SEQ ID NO: 1) of the alpha-isoform of survivin;
   (ii) a peptide of 27 consecutive amino acids located between positions 84 to 110 (SEQ ID NO: 2) of the alpha-isoform of survivin; and
   (iii) a peptide of 21 consecutive amino acids located between positions 122 and 142 (SEQ ID NO: 3) of the alpha-isoform of survivin;
(b) at least one immunostimulatory adjuvant; and
(c) at least one adjuvant capable of creating a depot effect.

The sequence of survivin is according to SEQ ID NO: 15.

In this embodiment, (b) may comprise an oligodeoxynucleotide-containing unmethylated CpG motif (CpG-ODN) and (c) may comprise a Montanide® adjuvant.

The immunogenic compositions of the present invention may be capable of inducing a T-cell mediated immune response against survivin. The T-cell mediated immune response may comprise inducing survivin-specific CD4⁺ and/or CD8⁺ T lymphocytes.

According to a second aspect of the present invention, there is provided an immunogenic composition according to the present invention, for use in the treatment of cancer. Preferably, the cancer over-expresses survivin.

According to a third aspect of the present invention, there is provided an immunogenic composition according to the present invention for use in the prophylactic or therapeutic immunization of a subject who has or may develop a cancer. Preferably, the cancer over-expresses survivin.

According to a fourth aspect of the present invention, there is provided an immunogenic composition according to the present invention, for use in the diagnosis, prognosis or therapeutic monitoring of a cancer in a subject. Preferably, the cancer over-expresses survivin.

According to a fifth aspect of the present invention, there is provided a kit of parts comprising:
i. at least one peptide derived from the alpha-isoform of survivin, or functional derivative thereof;
ii. at least one adjuvant; and

instructions for preparing an immunogenic composition according to the present invention.

According to a sixth aspect of the present invention, there is provided a method of preparing an immunogenic composition according to the present invention.

According to a seventh aspect of the present invention, there is provided a method of treating cancer, the method comprising administering the immunogenic composition according to the present invention. Preferably, the cancer over-expresses survivin.

According to an eighth aspect of the present invention, there is provided a method of prophylactic or therapeutic immunization of a subject who has or may develop cancer, the method comprising administering the immunogenic composition according to the present invention. Preferably, the cancer over-expresses survivin.

According to a ninth aspect of the present invention, there is provided a method of diagnosis, prognosis or therapeutic monitoring of a cancer in a subject, the method comprising administering the immunogenic composition according to the present invention. Preferably, the cancer over-expresses survivin.

### FIGURES

The present invention will be described with reference to the accompanying figures as follows:

FIG. 1 illustrates the frequency of CD4+ T cell precursors specific to peptides S1, S2 and S3 in a sample of 12 naïve healthy donors;

FIG. 2 illustrates the T cell immunogenicity of the SVX-1 vaccine (mixture of the three survivin LSPs: S1, S2 and S3) in different mouse strains (C57BL/6, CBA and BALB/c) and in HLA-A2/HLA-DR1 transgenic mouse model (Tg HLA-A2/DR1);

FIG. 3 illustrates that the T cell immunogenicity of the individual survivin polypeptides (S1, S2 and S3) are able to induce strong T cell responses of similar intensity in immunized BALB/c and Transgenic HLA-A2/DR1 mice;

FIG. 4 illustrates the T cell immunogenicity in vivo of the SVX-1 vaccine (peptides S1+S2+S3) formulated with various selected immuno-adjuvants;

FIG. 5 illustrates the therapeutic efficacy of SVX-1 against established colorectal tumour cells expressing the human Survivin (CT26-T);

FIG. 6 illustrates therapeutic efficacy of SVX-1 against established Renal cancer model expressing the human Survivin (Renca-T);

FIG. 7 illustrates the therapeutic efficacy of SVX-1 against established B cell lymphoma (A20) and the induction of long-term survival;

FIG. 8 illustrates the capacity of the SVX-1 vaccine to induce effective anti-tumor memory responses against B lymphoma tumor cells (A20);

FIG. 9 illustrates the percentage of CD8⁺ cells in the spleen of BALB/c mice before and one day after treatment with an anti-CD8 depleting antibody (at days 5 and 12) by flow cytometry staining, using anti-CD4 and anti-CD8 antibodies;

FIG. 10 illustrates the therapeutic efficacy of the SVX-1 vaccine against established MHC class I⁺/II⁻ colorectal tumour cells (CT26) in CD8-depleted mice;

FIG. 11 illustrates the therapeutic efficacy of the SVX-1 vaccine against MHC class I⁺/II⁺ tumour cells (A20) in CD8-depleted mice.

FIG. 12 illustrates spontaneous T-cell responses against SVX-1 peptides in the blood of healthy donors (A) and lung cancer patients (B).

### DETAILED DESCRIPTION

### Definitions

The term “peptide” refers to a series of amino acid residues, connected to one other typically by peptide bonds between the alpha-amino and carbonyl groups of the adjacent amino acids. A peptide may have any number of amino acid residues.

The term “long peptide” refers to peptide comprising 18 to 45 amino-acid residues. Long peptides are highly stable and can be synthesized efficiently in vivo, in vitro and in silico. They also allow efficient uptake by cells capable of processing said long peptide, present epitopes in the context of MHC-I or MHC-II, and provide a parallel and balanced stimulation of both CD4⁺ helper and CD8⁺ cytotoxic T cells. The length of the long peptides strongly favours peptide processing by ‘professional’ antigen-presenting cells (APC) to direct binding to major histocompatibility complex (MHC) on the cell surface. This minimizes the induction of immunological tolerance observed with the use of short CTL peptides (Zwaveling et al. 2002).

Antigen presenting cells and particularly professional antigen presenting cells such as dendritic cells are efficient in processing and presenting epitopes. They further comprise additional functionalities allowing efficient communication with T-cells which ultimately leads to improved induction and/or enhancement of said antigen specific T cell response (Quakkelaar and Melief 2012). Compared to recombinant proteins, long peptides can be rapidly and much more efficiently processed by dendritic cells (DCs), improving antigen (Ag) presentation and thus CD4⁺ and CD8⁺ T cell activation (Rosalia et al. 2013). In over 20 clinical trials, long synthetic peptide (LSP)-based vaccines were found to be safe, well tolerated, and showed promising clinical efficacy in patients with pre-neoplastic lesions as in patients infected with malaria or HPV (Kenter et al. 2009; de Vos van Steenwijk et al. 2014; van Poelgeest et al. 2013; Zeestraten et al. 2013; Vermeij et al. 2012; Audran et al. 2009).

The term “oligopeptide” refers to a peptide comprising 2 to 20 amino-acid residues.

The term “polypeptide” refers to a continuous, unbranched peptide chain.

The term “lipopeptide” refers to a peptide that has a lipid attached to it.

The term “expression vector” or “expression construct”, refers to a host, usually a plasmid or virus, designed for protein expression in cells. The vector is used to introduce a specific gene into a target cell, and may use or stimulate the cell's own mechanism for protein synthesis to produce the protein encoded by the gene.

The term “nucleotide” refers to monomer that form the building blocks of nucleic acids e.g. DNA, RNA.

The term “polynucleotide” refers to a biological polymer comprising a chain of nucleotide monomers that are covalently bonded, for example, DNA and RNA.

The term “concatenated” in the context of concatenated peptides refers to two or more peptides joined, for example, end-to-end, directly or via a linker, another entity, a scaffold and/or a combination therapy.

The term “linker” refers to a peptide sequence that may occur between protein domains and may be synthetic or natural. Linkers are often composed of flexible residues like glycine and serine so that the adjacent protein domains are free to move relative to one another. Longer linkers are used when it is necessary to ensure that two adjacent domains do not sterically interfere with one another.

If desired, the individual amino acid sequences of the components of the fusion proteins can be produced and joined by a linker. Suitable peptide linker sequences may be chosen based on the following factors: (1) their ability to adopt a flexible extended conformation, (2) their ability to adopt a secondary structure that could interact with functional epitopes of the first and second polypeptides, (3) the lack of hydrophobic or charged residues that might react with the polypeptide functional epitopes, (4) the ability to increase solubility, and (5) the ability to increase sensitivity to processing by antigen-presenting cells. Such linkers can be any amino acid sequence or other appropriate link or joining agent.

Linkers useful in the invention include linkers comprising a charged amino acid pair such as KK or, linkers sensitive to cathepsin and/or other trypsin-like enzymes, thrombin or Factor Xa, or linkers which result in an increase in solubility of the peptide. Specific examples of linkers include those linkers that contain Gly, Asn and Ser residues. The linker sequence may be from 1 to about 150 amino acids in length or even longer.

The term “derivative” in the context of a derivative of a peptide refers to any peptide-containing compound that is derived from a similar or the same peptide, including but not limited to: oligopeptide, polypeptide, lipopeptide, neuropeptide, neuropeptide, proteose etc.

The term “functional derivative” refers to any peptide derivative or any construct or precursor capable of expressing the peptide, polypeptide or peptide-containing compound, for example, an expression vector or polynucleotide.

It is known in the art that peptides may be “labelled” or “complexed” such that the resulting peptide conjugates can be used as sensors, markers or chelating agents for medical or analytical purposes. For example, the peptides of the present invention or functional derivatives thereof may be labelled with anti-CD14, -CD86, -HLA-DR, -CD80 (Becton Dickinson), -Cd1a, -HLA-ABC, -CD83 and -CD16 (Beckman Coulter) antibodies, conjugated to a fluorochrome. Labelled peptides can be prepared either by modifying isolated peptides or by incorporating the label during solid-phase synthesis.

The term “adjuvant” refers to a pharmacological or immunological component that potentiates and/or modulates the immune response to an antigen, but would normally not provide immunity alone. An “immunostimulatory” adjuvant or “immune-potentiating” adjuvant has the capacity to stimulate or improve an immune response e.g. by activating the innate immune system. Suitable adjuvants would be known to those skilled in the art. The present invention has identified adjuvants such as unmethylated cytosine-guanosine dinucleotide (CpG) motifs and granulocyte macrophage colony-stimulating factor (GM-CSF) to be suitable immunostimulatory adjuvants.

Some adjuvants can act as a “depot” for an antigen, trapping antigens e.g. at the injection site, and providing slow release over a period of time in order to modulate the stimulation of the immune system. An adjuvant that is “capable of creating depot effect” may be any antigen that can act as a depot. For example, alum, emulsion based formulations, mineral oil, non-mineral oil, and oil-in-water emulsions are all examples of adjuvants capable of creating a depot effect. In particular, the Seppic ISA series of Montanide® adjuvants, including but not limited to MF-59, ISA 51 VG and AS03 have been identified as suitable adjuvants that are capable of creating a depot effect.

“Montanide®” adjuvants would be well known to those skilled in the art. They belong to a family of oil-based adjuvants that have been used in experimental vaccines in mice, rats, dogs and cats using, for example, natural, recombinant or synthetic antigens. In humans, Montanide has been used in trial vaccines against HIV, malaria and breast cancer. There are several different types of Montanides including ISA, 50V 20G and 720. Emulsions of Montanide ISA, 50V and 720 are composed of metabolizable sequence based oil with a mannide mono-oliate emulsifier. At the time of writing, the compositions of the Montanides® were proprietary.

“MF-59” is a submicron oil-in-water emulsion which contains squalene (around ˜2.5% (vol/vol)) and varying amounts of muramyl tripeptide phosphatidyl-ethanolamine (MTP-PE).

“ISA 51 VG” is a water-in-oil (w/o) emulsion comprising a surfactant mannide monooleate which contains vegetable-grade (VG) oleic acid derived from olive oil.

“AS03” is an oil-in-water emulsion comprising squalene (around ˜2.5% (vol/vol)), L-a-tocopherol and polysorbate 80.

The term “T cell epitope” refers to a peptide that can bind to a MHC class I or II receptor, forming a ternary complex that can be recognized by a T cell bearing a matching T-cell receptor that binds to the MHC/peptide complex with appropriate affinity. CD8⁺ T cells recognize antigenic peptides of 8 to 10 amino acids presented by MHC class I molecules whereas the CD4⁺ T cells recognize peptides of 15 to 20 amino acids presented by MHC class II molecules; in humans, they are called HLA I and II molecules, for Human Leukocyte Antigen (HLA) class I and II molecules. The principal activation pathway takes place via the professional antigen-presenting cells (APCs) (B cells, dendritic cells, macrophages, in addition to thymic epithelial cells). Alternatively, the recognition may take place directly (i.e. the tumour itself presents these peptides to the T lymphocytes).

The antigenic peptides, called CD4 and CD8 T epitopes, result from the proteolytic degradation, of the antigens by the antigen presenting cells. They have varying lengths and have a sequence, which makes them capable of binding to the HLA I or II molecules. In the case of peptides that bind to MHC class II molecules, the same peptide and the corresponding T cell epitope may share a common core segment, but differ in the overall length due to flanking sequences of differing lengths upstream of the amino-terminus of the core sequence and downstream of its carboxy-terminus, respectively. MHC class II receptors have a more open conformation. Peptides bound to MHC class II receptors are not completely buried in the structure of the MHC class II molecule peptide-binding cleft as they are in the MHC class I molecule peptide-binding cleft.

In humans there are three different genetic loci that encode MHC class I molecules: HLA-A, HLA-B and HLA-C. HLA-A*01, HLA-A*02, and HLA-A*11 are examples of different MHC class I alleles that can be expressed from these loci. There are three different loci in the human genome for MHC class II genes: HLA-DR, HLA-DQ, and HLA-DP. MHC class II receptors are heterodimers consisting of an alpha and a beta chain, both anchoring in the cell membrane via a transmembrane region. HLA-DRB 1*04, and HLA-DRB1*07 are two examples of different MHC class II beta alleles that are known to be encoded in these loci. Class II alleles are very polymorphic, e.g. several hundred different HLA-DRB1 alleles have been described. However, CD4+ T cell responses often described in cancer research are restricted to HLA class II molecule encoded by the HLA-DR sublocus. Therefore, for therapeutic and diagnostic purposes a peptide that binds with appropriate affinity to several different HLA class II receptors is highly desirable. A peptide binding to several different HLA class II molecules is called a “promiscuous”.

The term “survivin” refers to the isoforms of survivin: alpha isoform, Survivin-2α, Survivin-2B, Survivin-Δ3Ex, Survivin-3B, and Survivin-3α. These isoforms can be derived from any mammal; preferably, it is human isoforms. The alpha isoform of survivin is the survivin consisting of 142 amino acids; positions are shown with reference to the human sequence (SEQ ID NO: 15, Genbank AAC51660 or SwissProt 015 392).

The term “cancer” refers to a cancer non-limited to: breast, liver, colon, lung, ovary, uterus, esophagus, stomach, pancreas, liver and prostate, melanoma, Hodgkin's disease, non-Hodgkin lymphoma, leukaemia, myelodysplastic syndrome with refractory anaemia, neuroblastomas, pheochromocytomes, soft tissue sarcomas, brain tumours and/or virus associated cancers e.g. Human papilloma virus (HPV), Epstein-Barr Virus (EBV), hepatitis B, hepatitis C, human immunodeficiency virus (HIV), Kaposi Sarcoma.

A cancer “overexpressing survivin” refers to a cancer associated with overexpression of survivin i.e. a level of survivin above what would be expected in normal adult tissue.

The term “therapeutic monitoring” refers to a clinical practice of measuring the concentration of specific drugs at designated intervals e.g. in the bloodstream of a subject, primarily with an aim to maintain a constant concentration, thereby optimizing individual dosage regimens. In some cases, a subject may be monitored for one or more weeks or in other cases one or more months. The response of the subject to the therapy may be monitored and the therapy adjusted accordingly, for example, the type or combination of therapies or drugs, mode of administration and the dosage regime.

It would be appreciated by the skilled person that appropriate dosage regimes may depend on factors such as the body weight of the patient, the stage of the cancer to be treated, and the type of cancer.

The term “predominant HLA II molecule in the Caucasian population” or “predominant HLA II molecule” is intended to mean an HLA II molecule comprising a beta chain encoded by an allele at a frequency greater than 5% in the Caucasian population, as specified in Table I below. Some of the HLA II molecules predominant in the Caucasian population, in particular HLA-DP401 and HLA-DP402 molecules, are also predominant in other populations (South America, India, Japan, Africa). Therefore, the long peptides of the invention are not restricted for use in the Caucasian population, and they can also be used to immunize individuals from countries other than those in North America and Europe, where such molecules HLA II are predominant.

The present invention provides vaccine compositions and formulations, particularly for use in inhibiting growth of cancer cells that over-express survivin. The compositions of the invention elicit strong antitumor cell-mediated immunity capable of inhibiting the growth of tumours that contain survivin expressing cancer cells.

The term “over-expression” in relation to survivin expression refers to cells that express greater levels of survivin when compared to healthy/normal cells.

Survivin represents a particularly attractive target for antitumor immunization due to its restricted overexpression and vital functions in most human tumours and its capacity to induce tumour-specific CD4⁺ and CD8⁺ T cell responses.

A cancer vaccine targeting survivin can be used to treat various malignancies, as survivin is expressed in the majority of tumours. In addition, the use of an antigen essential to tumour survival, such as survivin, as a target for antitumor immunization, makes it possible to avoid problems of tumours evading recognition by the immune system.

Antitumor vaccines targeting survivin have been evaluated in clinical trials in patients suffering from various malignancies (e.g. myeloma, non-small-cell lung cancer, melanoma, ovarian cancer, bladder, and renal cell and prostate carcinoma) demonstrating that immune responses against survivin can be induced in cancer patients without raising safety concerns (Otto et al. 2005; Berntsen et al. 2008; Trepiakas et al. 2010; Ellebaek et al. 2012; Hobo et al. 2013; Rittig et al. 2011; Wobser et al. 2006; Rapoport et al. 2014; Widenmeyer et al. 2012; Becker et al. 2012; Lennerz et al. 2014).

The lack of success in the prior art to provide viable vaccine candidates targeting survivin is thought to be related to an inappropriate design and/or composition of these vaccines. The majority used recombinant proteins, DNA, or short Survivin CD8⁺ T cell epitopes inducing tumour-antigen-specific cytotoxic T lymphocytes with a very low frequency (of the order of 10⁻⁴ to 10⁻⁷ of the CD8+ T cells). The lack of success may also be related to an inappropriate vaccine formulation to generate effective antitumor T cell responses with recombinant and peptide-based vaccines.

The present applicants have developed a novel cancer vaccine which surprisingly induces both an effective and long-term immune responses against tumours overexpressing survivin.

The invention provides an immunogenic composition comprising:
(a) at least one peptide derived from the alpha-isoform of survivin, or functional derivative thereof;
(b) at least one immunostimulatory adjuvant; and
(c) at least one adjuvant capable of creating a depot effect.

The variants of the survivin proteins and fragments thereof may also include peptides comprising non-traditional amino acid residues. For example, the MtrE peptides and fragments thereof may include residues in the “D configuration” or amino acids that do not normally occur in proteins, such as but not limited to citrulline, ornithine, hypusine, selenocysteine a-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, norleucine, norvaline, hydroxyproline, sarcosine, cysteic acid, t-butylglycine, t-butylalanine, phenylglycine, cyclohexylalanine, β-alanine, fluoro-amino acids, designer amino acids such as β-methyl amino acids, Ca-methyl amino acids, Na-methyl amino acids, PNA's and amino acid analogs in general. Furthermore, the amino acid can be D- or L-isoform.

In some embodiments, the peptides derived from the alpha-isoform of survivin may be isolated peptides. The isolated proteins of the present invention can occur in any in vitro or in vivo setting. For example, a cell containing a vector that encodes a survivin protein of the present invention encompasses the term “isolated protein” as used herein. Thus, a survivin protein present in a cell that does not normally express survivin, regardless of how it was introduced into the cell, is also encompassed within the term “isolated protein” as used herein.

However, a nucleic acid contained in a clone that is a member of a library, e.g., a genomic or cDNA library, that has not been isolated from other members of the library, e.g., in the form of a homogeneous solution containing the clone and other members of the library, or a chromosome isolated or removed from a cell or a cell lysate, e.g., a “chromosome spread,” as in a karyotype, is not “isolated” for the purposes of the invention. As discussed further herein, isolated nucleic acid molecules according to the present invention may be produced naturally, recombinantly, or synthetically.

Of course, the isolated survivin proteins or fragments described herein can be purified or substantially purified. As used herein, the term “purified” when used in reference to a protein or nucleic acid, means that the concentration of the molecule being purified has been increased relative to other molecules associated with it in its natural environment, or environment in which it was produced, found or synthesized. One of skill in the relevant art would recognise that these “other molecules” might include proteins, nucleic acids, lipids and sugars but generally do not include water, solvents, buffers, and reagents added to maintain the integrity or facilitate the purification of the protein being purified. For example, even if a protein is diluted with an aqueous solvent during affinity chromatography, the proteins are purified by this chromatography if other naturally associated molecules do not bind to the column and are separated from the proteins or fragments of interest. According to this definition, a proteins or fragments may be 5% or more, 10% or more, 20% or more, 30% or more, 40% or more, 50% or more, 60% or more, 70% or more, 80% or more, 90% or more, 95% or more, 98% or more, 99% or more, or 100% pure when considered relative to its contaminants.

The skilled person would be familiar with methods such as ‘gap penalty’ for sequence alignments.

The composition of present invention provides long peptides derived from the wild type human survivin sequence, wherein the peptides encompass multiple Survivin derived CD4+ T cell epitopes capable of inducing survivin-specific CD4+ T cell responses and to be presented by several HLA class II molecules predominant in the Caucasian population. The said long polypeptides are more effective than short peptides or recombinant protein at generating strong and long-term human T cell responses against survivin-expressing cancer cells.

The immunogenic composition is also provided for use in the treatment of a cancer, for use in the prophylactic or therapeutic immunization of a subject who has or may develop a cancer and/or for use in the diagnosis, prognosis or therapeutic monitoring of a cancer in a subject. Preferably, the cancer over-expresses survivin.

The immunogenic or vaccine compositions comprise one or more peptides derived from the alpha-isoform of survivin (Table I). The peptide(s) in (a) may be selected from the group consisting of:
(i) the peptide of 18 consecutive amino acids located between positions 17 and 34 (SEQ ID NO:1) of the alpha-isoform of Survivin, referred as S1 peptide, which include the 15 amino acid peptides in positions 20 to 34 (SEQ ID NO:4) and 17 to 31 (SEQ ID NO:5) of the alpha-isoform of survivin,
(ii) the peptides of 27 consecutive amino acids located between positions 84 and 110 (SEQ ID NO:2) of the alpha-isoform of Survivin, referred as S2 peptide, which include the 15 amino acid peptides in positions 84 to 98 (SEQ ID NO:6), 90 to 104 (SEQ ID NO:7), 93 to 107 (SEQ ID NO:8) or 96 to 110 (SEQ ID NO:9) of the alpha-isoform of survivin, and
(iii) the peptides of 21 consecutive amino acids located between positions 122 and 142 (SEQ ID NO:3) of the alpha-isoform of Survivin, referred as S3 peptide, which include the 15 amino acid peptides in positions 128 to 142 (SEQ ID NO:11) of the alpha-isoform of survivin,

The peptides in (i), (ii) and (iii) are capable of generating T cell mediated immune responses against survivin.

TABLE I
Sequences of the Survivin derived peptides comprise in the SVX-1 vaccine

| SEQ | | | | |
|---|---|---|---|---|
| ID No. | Name | Size° | Positions* | Sequence |
| 1 | S1 | 18 | 17-34 | H R I S T F K N W P F L E G C A C T |
| 2 | S2 | 27 | 84-110 | C A F L S V K K Q F E E L T L G E F L K L D R E R A K |
| 3 | S3 | 21 | 122-142 | K E F E E T A K K V R R A I E Q L A A M D |

Legend of Table I:
°Number of amino acids
*The positions are numbered with reference to the sequence of human Survivin of 142 amino acids (SEQ ID NO: 15, Swissprot #015392).

Each of said peptides contained promiscuous Survivin-derived CD4+ T cell epitopes able to be presented by several HLA class II molecules predominant in the Caucasian population, namely HLA-DR1, HLA-DR3, HLA-DR4, HLA-DR7, HLA-DR1, HLA-DR13, HLA-DR15, HLA-DRB3, HLA-DRB4, HLA-DRB5 and HLA-DP4 (WO 2007/036638; Wang et al. 2008) (Table II).

SEQ ID NO:1 (HRISTFKNWPFLEGCACT) is a 18 amino acid peptide consisting of wild type Survivin amino acids 17-34, referred as S1 peptide, which includes peptides of 15 amino acids located in positions 20 to 34 (SEQ ID NO: 4) and 17 to 31 (SEQ ID NO: 5) of the alpha-isoform of Survivin, each containing a promiscuous CD4 T cell epitope.

SEQ ID NO:2 (CAFLSVKKQFEELTLGEFLKLDRERAK) is a 27 amino acid peptide consisting of wild type Survivin amino acids 84-110, referred as S2 peptide, which includes peptides of 15 amino acids located in positions 84 to 98 (SEQ ID NO:6), 90 to 104 (SEQ ID NO:7), 93 to 107 (SEQ ID NO:8), and 96 to 110 (SEQ ID NO:9) of the alpha-isoform of Survivin, each containing a promiscuous CD4 T cell epitope.

SEQ ID NO:3 (KEFEETAKKVRRAIEQLAAMD) is a 21 amino acid peptide consisting of wild type Survivin amino acids 122-142, referred as S3 peptide, which includes peptides of 15 amino acids located in positions 122 to 142 (SEQ ID NO:10), and 128 to 142 (SEQ ID NO:11) of the alpha-isoform of Survivin, each containing a CD4 T cell epitope.

In one embodiment, the vaccine composition comprises the group of long peptides derived from the alpha isoform of Survivin, consisting of peptide 17-34 (S1 peptide, SEQ ID NO: 1), peptide 84-110 (S2 peptide, SEQ ID NO: 2) and peptide 122-142 (S3 peptide, SEQ ID NO: 3), and referred to herein as the SVX-1 vaccine.

As used herein, the term “SVX-1 peptides” is intended to mean a group of long peptides, consisting of peptide 17-34 (SEQ ID NO: 1), peptide 84-110 (SEQ ID NO: 2) and peptide 122-142 (SEQ ID NO: 3), and are present in the SVX-1 vaccine.

TABLE II
Position and amino acid sequence of the Survivin derived CD4⁺ T-cell
epitopes contained in the SVX-1 vaccine

| LSP | | SEQ ID | | |
|---|---|---|---|---|
| name | Position* | No. | Sequence | HLA class II restriction° |
| S1 | 17-31 | 4 | HRISTFKNWPFLEGC | HLA-DRB1*0401, *0701, *1501, HLA- |
| | | | | DRB4*0101, HLA-DRB5*0101, HLA- |
| | | | | DP4*0201 |
| | 20-34 | 5 | STFKNWPFLEGCACT | HLA-DRB1*0401, *0701, *1101, HLA- |
| | | | | DP4*0201 |
| S2 | 84-98 | 6 | CAFLSVKKQFEELTL | ND |
| | 90-104 | 7 | KKQFEELTLGEFLKL | HLA-DRB1*0701, *1101, *1501, HLA- |
| | | | | DRB4*0101, HLA-DRB5*0101, HLA- |
| | | | | DP4*0201 |
| | 93-107 | 8 | FEELTLGEFLKLDRE | HLA-DRB1*1101, HLA-DP4*0201 |
| | 96-110 | 9 | LTLGEFLKLDRERAK | HLA-DRB1*0401, *0701, HLA-DRB4*0101, |
| | | | | HLA-DP4*0201 |
| S3 | 122-136 | 10 | KEFEETAKKVRRAIE | ND |
| | 128-142 | 11 | AKKVRRAIEQLAAMD | HLA-DRB1*1501, HLA-DRB5*0101 |

Legend of Table II
*The positions are numbered with reference to the sequence of human Survivin of 142 amino acids (SEQ ID NO: 15, Swissprot #015392)
°HLA class II restriction of Survivin derived CD4⁺ T-cell epitopes as described in the WO2007036638 and in Wang et al. (Wang et al. 2008)
ND: Not determined

The capacity of one or more SVX-1 peptides (S1, S2, and S3) of the present invention has been demonstrated to induce strong CD4⁺ T-cell responses in vitro with peripheral blood mononuclear cells (PBMCs) from healthy donors displaying various HLA class II types. A high frequency of spontaneous CD4⁺ T-cell precursors specific to the SVX-1 vaccine circulating in humans has also been identified. Altogether, this predicts a relatively high CD4⁺ T cell immunogenicity of the SVX-1 vaccine and the individual polypeptides in humans, irrespective of the individual's HLA type.

The present invention provides vaccine formulations wherein the peptides in (a) are combined with adjuvants (b) and (c). Said formulations comprise one or more immunostimulatory adjuvants which may comprise an immunostimulatory oligonucleotide containing at least one unmethylated CpG motif.

The formulations further comprise an adjuvant that creates a depot effect selected from but not restricted to the group consisting of alum and emulsion based formulations including mineral oil, non-mineral oil, and O/W emulsions such as Seppic ISA series of Montanide adjuvants, MF-59, and ASO03.

Immunostimulatory oligonucleotides containing unmethylated CpG motifs (“CpG ODN”) are known in the art as being adjuvants when administered by both systemic and mucosal routes. CpG is an abbreviation for cytosine-guanosine dinucleotide motifs present in DNA. Historically, it was reported that DNA extracts from Mycobacter tuberculosis can activate NK cells and exert an anti-tumor effect (Tokunaga et al. 1984). Subsequent works showed that the immunogenic properties were due to the presence within the bacterial DNA of CpG sequences (Yamamoto et al. 1992; Krieg et al. 1995), which are suppressed and methylated in vertebrate DNA (Bird et al. 1987).

CpG ODNs are recognized by TLR9, which is expressed exclusively on human B cells and plasmacytoid dendritic cells (pDCs), thereby inducing Th1-dominated immune responses (Coffman, Sher, and Seder 2010).

Examples of oligonucleotides that may be used have the following sequences. The sequences may contain phosphorothioate modified inter-nucleotide linkages.

OLIGO 1:
(SEQ ID NO: 12)
TAA ACG TTA TAA CGT TAT GAC GTC AT (Litenimod)
OLIGO 4:
(SEQ ID NO: 13)
TCG TCG TTT TGT CGT TTT GTC GTT (CpG 2006)
OLIGO 5:
(SEQ ID NO: 14)
GGG GAC GAC GTC GTG TGG GGG GG (CpG 2336)

Adjuvants such as alum and O/W emulsions function as delivery systems by generating depots that trap antigens at the injection site, providing slow release in order to continue the stimulation of the immune system. These adjuvants enhance the antigen persistence at the injection site and increase recruitment and activation of antigen presenting cells (APCs). Particulate adjuvants such as alum also have the capability to bind antigens to form multi-molecular aggregates which will encourage uptake by APCs.

The novel combinations of adjuvants enhance and polarize T cell responses induce with long peptide-based vaccines towards Th1 profile, thus directing their adaptive immune responses.

The inventors have demonstrated that the formulation of the long peptides of the SVX-1 vaccine with an immunostimulatory oligonucleotide containing unmethylated CpG motifs alone or emulsified in a O/W emulsion, significantly improve their capacity to induce in vivo strong T cell responses secreting high amount of interferon (IFN)-γ, characteristic of a Th1 profile (see Examples).

The T cell immunogenicity of the SVX-1 vaccine was found to be significantly higher when formulated with IC31, CpG, AFPL1 or Poly-ICLC compared to Montanide and MPLA. In addition, the combination of CpG with GM-CSF but in particular with Montanide (ISA 51 VG) significantly increased the immunogenicity of the SVX-1 vaccine.

Stimulation of a CD4 and/or a CD8 T Cell Response and Therapeutic Efficacy

The inventors have demonstrated the capacity of the SVX-1 vaccine and the individual peptides to generate strong T-cell responses secreting high amount of IFN-γ in different mouse strains and in an HLA-A2/DR1 transgenic mouse model, expressing the human HLA class I and II molecules. The inventors have also demonstrated the capacity of the SVX-1 vaccine to significantly impede the growth of various established mouse tumour graft models, expressing only MHC class I molecules or both MHC class I and class II molecules, associated with its capacity to generate strong and long lasting CD4+ but also CD8+ T-cell responses specific to the SVX-1 peptides.

Finally, spontaneous T-cell precursors specific to the SVX-1 peptides have been detected in cancer patients but not in healthy donors, indicating the absence of immune tolerance against the peptides of the present invention in cancer patients.

Therefore, it is expected that the vaccine composition of the present invention can be used for improved human anti-tumoral T cell responses against survivin-expressing cancer cells, and thus can be used for prophylactic, ameliorating and/or curative treatment of cancer diseases.

Peptide Preparation

The peptides described herein can be produced by any technique known to those skilled in the art or by subsequently developed techniques. For example, they can be synthesized using standard direct peptide synthesizing techniques (Birr 1985), such as via solid-phase synthesis (Merrifield 1963; Barany, Kneib-Cordonier, and Mullen 1987). Alternatively, a gene encoding the desired long peptides can be subcloned into an appropriate expression vector using well-known molecular genetic techniques. The peptides can then be produced by a host cell and isolated from the cell. Any appropriate expression vector (Pouwels, Enger-Valk, and Brammar 1985) and corresponding suitable host cells can be employed for production of the desired peptide. Expression hosts include, but are not limited to, bacterial species, mammalian or insect host cell systems including baculovirus systems (Luckow and Summers 1988), and established cell lines such 293, COS-7, C127, 3T3, CHO, HeLa, BHK, etc.

Once it is manufactured and suitably isolated, the inventive polypeptides may be substantially purified by preparative high performance liquid chromatography or other comparable techniques available in the art. The composition of the synthetic peptides can be confirmed by a technique for amino acid composition analysis.

A further aspect of the invention provides a kit of parts comprising:
i. at least one peptide derived from the alpha-isoform of survivin, or functional derivative thereof;
ii. at least one adjuvant; and

instructions for preparing of an immunogenic composition.

Further aspects of the invention provide methods of treating cancer. The method may comprise administering to an individual diagnosed with or suspected of having a survivin expressing cancer a formulated vaccine of the invention in an amount effective to inhibit growth of the survivin expressing cancer cells in the individual. Inhibition of growth as used herein could include for example, a reduction of the size of an existing tumour or reduced growth of the tumour.

The method of treatment may comprise prophylactic or therapeutic immunization of a subject, or diagnosis, prognosis or therapeutic monitoring of a cancer in a subject.

### Example 1

Prediction of the CD4⁺ T Cell Immunogenicity of the SVX-1 Vaccine and the Individuals Survivin Derived LSPs (S1, S2, and S3) in Human

The ability of the 3 native Survivin derived long Synthetic peptides (LSP S1, S2 and S3) and the mixture of the 3 LSPs to induce in vitro stimulation of specific CD4⁺ T cells was evaluated from blood samples of healthy individuals (non-tumor-bearing). The aim is to assess the ability of these peptides to recruit CD4⁺ lymphocyte precursors while they are in a naïve individual to a very low frequency, and thus to predict the immunogenicity and immunoprevalence of the mix of peptides and the individual peptides in human.

1) Materials and Methods

a) Peptides Manufacturing

Two pilot batches of the LSPs covering the sequences 17-34 (S1), 84-110 (S2) and 114-122 (S3) of the native Survivin protein were synthetized by solid phase synthesis using the fluorenylmethoxycarbonyl-t-butyl strategy.

The sequences of the three LSPs (S1, S2 and S3) are given in the Table I and in the accompanying sequence listing.

After deprotection and cleavage, the peptides were purified by reverse phase HPLC (Vydac C18 column, Interchip). Yield of manufacturing, purity, solubility, and molecular weight of S1, S2, and S3 final products were determined by HPLC (High-performance liquid chromatography) and determination of amino acid composition after total acid hydrolysis, and molecular mass controlled by mass spectrometry (ES-MS).

As shown in the Table III, all the survivin long peptides were successfully manufactured, with a good yield, purity (>90%) and solubility, demonstrating the ease of production of these peptides.

TABLE III
Biochemical characterization of the Survivin
peptides (SEQ ID Nos. 1 to 3)

| | Final | | | | |
|---|---|---|---|---|---|
| | Product | Mmol | | | Determined |
| | Weight | (out) | Yield | Purity | Molecular |
| Product | (mg) | Corrected | (mol %) | (% area/area) | Weight |
| Batch 1 | | | | | |
| S1 | 121 | 0.057 | 16 | 98.5 | 2109.2 |
| S2 | 72 | 0.032 | 9 | 96.2 | 3198.1 |
| S3 | 177 | 0.092 | 26 | 95.9 | 2462.6 |
| Batch 2 | | | | | |
| S1 | 46 | 0.022 | 13 | 95.0 | 2109.1 |
| S2 | 106 | 0.033 | 10 | 90.8 | 3197.9 |
| S3 | 100 | 0.092 | 26 | 97.1 | 2462.4 |

b) Individuals Tested

Blood samples from twelve healthy donors were collected at the Etablissement Franrais du Sang (EFS, Rungis, France) as residual buffy-coat preparations from anonymous healthy donors after informed consent and following the guidelines of the EFS. Peripheral blood mononuclear cells (PBMCs) from the healthy donors were separated on a Ficoll gradient (Ficoll-Hypaque, Sigma-Aldrich) and the HLA-DR phenotype in donors was determined by SSP 5 using the Olerup SSP™ HLA-DRBL (Olerup SSP AB) kit. The HLA-DRBL phenotype of these donors and their characteristics (gender, age, and serology) as provided by EFS, are presented in Table IV.

TABLE IV

| Normal | Characteristics and HLA typing of the donor tested | | | | | | |
|---|---|---|---|---|---|---|---|
| Donor | DRB1 | | Second DR molecule | | Gender | Age | Serology * |
| D1 | DRB1*0701 | DRB1*1101 | DRB3 | DRB4 | M | 53 | Neg |
| D2 | DRB1*0102 | DRB1*0301 | DRB3 | | F | 20 | Neg |
| D3 | DRB1*0401 | DRB1*0701 | DRB4 | | F | 22 | Neg |
| D4 | DRB1*1301 | DRB1*1401 | DRB3 | | M | 47 | Neg |
| D5 | DRB1*0401 | DRB1*0701 | DRB4 | | M | 42 | Neg |
| D6 | DRB1*0102 | DRB1*1201 | DRB3 | | F | 26 | Neg |
| D7 | DRB1*0103 | DRB1*1501 | DRB5 | | M | 49 | Neg |
| D8 | DRB1*0401 | DRB1*1501 | DRB4 | DRB5 | M | 51 | Neg |
| D9 | DRB1*0701 | DRB1*1601 | DRB4 | DRB5 | F | 56 | Neg |
| D10 | DRB1*1104 | DRB1*1501 | DRB3 | DRB5 | F | 22 | Neg |
| D11 | DRB1*0801 | DRB1*1301 | DRB3 | | M | 24 | Neg |
| D12 | DRB1*03 | DRB1*04 | DRB3 | DRB4 | M | 27 | Neg |

Legend of Table IV
*Negative serology given by EFS: CMV, QPA, HIV, HTLV, HBs, AgHBs, cHBs, HCV, Pal RAE and Hmo Hma Sy.

b) Establishment of a Collection of CD4⁺ T Lymphocytes and Autologous mDC Obtained from Blood of Characterized Healthy Donors

Peripheral blood mononuclear cells (PBMCs) were separated on a Ficoll gradient (Ficoll-Hypaque, Sigma-Aldrich). The PBMCs were then cultured in AIMV medium (Life Technologies; 10′ cells/ml) and incubated in flasks, in an incubator at 37° C. with 5% C02. After 2 hours, non-adherent cells (NA cells) were harvested, frozen and stored as several aliquots in liquid nitrogen, according to a standard procedure, and until used for CD4⁺ T cell isolation and ELISpot analyses.

The adherent cells (Monocytes) were incubated for 5 to 6 days, in AIMV medium supplemented with 1000 units/ml of recombinant human GMCSF and 1000 U/ml of recombinant human IL-4 (rh-GM CSF and rh-IL-4; Tebu), to generate immature dendritic cells (imDCs). The imDCs were subsequently cultured for 2 days in the presence of 1 ug/ml of LPS (Sigma), 1000 U/ml of rh-IL-4 and 1000 U/ml of rh-GM CSF, so as to induce their maturation.

The quality of the DC preparations was evaluated by flow cytometry (FACScalibur flow Cytometer™, Becton Dickinson) assisted by the Cell Quest Pro™ software (Becton Dickinson). To this end, the DCs were labeled with anti-CD14, -CD86, -HLA-DR, -CD80 (Becton Dickinson), -Cd1a, -HLA-ABC, -CD83 and -CD16 (Beckman Coulter) antibodies, conjugated to a fluorochrome.

The CD4⁺ T lymphocytes were isolated from the thawed NA cells by positive selection using both anti-CD4 monoclonal antibody coupled to magnetic microbeads and magnetic cell sorting, as recommended by the manufacturer (Myltenyi Biotech kit). The cells were used immediately to induce CD4⁺ T cell lines.

c) Generation of Ag-Specific CD4⁺ T Cell Lines from Healthy Donors

Mature dendritic cells (mDCs; 5×10⁵ cells in 1 ml) were incubated with a mixture of the 3 Survivin peptides (10 μg of each peptide) or a pool of 10 well-known immunogenic peptides (Positive peptides) in IMDM medium (Invitrogen) supplemented with glutamine (24 mM, Sigma), asparagines (55 mM, Sigma), arginine (150 mM, Sigma), penicillin (50 IU/ml, Invitrogen), streptomycin (50 mg/ml, Invitrogen) and 10% of human AB serum, herein after referred to as complete IMDM medium, for 4 hours at 37° C.

CD4⁺ T cell lines were generated by incubating 10⁵ naïve CD4⁺ T lymphocytes with 10⁵ loaded autologous mDCs previously washed (ratio 1:10), in 200 l per round-bottom micro well of complete IMDM medium containing 1000 U/ml of IL-6 (R&D systems) and 10 ng/ml of IL-12 (R&D systems). CD4⁺ T cell lines were incubated at 37° C. in 5% CO2. The CD4⁺ T lines were restimulated once a week with fresh autologous mDCs loaded with peptides, supplemented by 10 U/mL IL2 and 5 ng/mL IL7. After three rounds of in vitro stimulation (Day 7 (D7), D14 and D21) the specificity of the CD4⁺ T cell lines was investigated by measuring the production of IFN-γ using ELISpot assays one week after the last stimulation (D28).

d) Analysis of the Specificity of the Lines by ELISpot Assays

The CD4⁺ T cells amplified in vitro were harvested at day 28, washed in IMDM medium and their specificity for the individual peptides S1, S2 and S3 was independently assessed in duplicate using IFN-γ ELISpot assays.

Anti-IFN-γ human monoclonal antibodies 1-DIK (MABTECH), diluted to 10 μg/ml in PBS buffer, were adsorbed onto nitrocellulose plates (Multiscreen HA; Millipore) for 1 hour at 37° C. The plates were subsequently washed with PBS and then saturated with complete IMDM medium (100 μl/Well), for 1 h at 37° C.

The antigen-presenting cells were autologous PBMCs. The antigen-presenting cells (10⁵ autologous PBMCs) and the CD4⁺ T lymphocytes to be tested (10⁴ CD4⁺ T lymphocytes) were subsequently added to the plates and incubated for 24 h at 37° C., in the presence or absence of a single peptide (2 ug of S1, S2 or S3 peptide). The peptides were added directly to the plates. After three successive washes with water, and then with PBS buffer-0.05% Tween and, finally, with PBS alone, 100 μl of biotin-conjugated anti-IFN-γ secondary antibody (7-B6-1 biotin, MABTECH), diluted to 0.25 μg/ml in PBS containing 1% BSA, were added to each Well. After incubation for one hour, the plates were washed again and incubated with 100 l/well of extravidin-phosphatase (E-2636, SIGMA), diluted to 1/6000. After washing of the plates in PBS buffer, 100 μl of NBT/BCIP substrate (B-5655, SIGMA), diluted in water (one tablet in 10 ml of water), and were distributed into each well. The Immuno enzymatic revelation was stopped after approximately 10 minutes by thorough rinsing of the plates in water, and the coloured spots were counted using an automatic reader (ELISpot reader system, AID). One spot was referred to as one CD4⁺ T cell that produced IFN-γ. The lines were considered to be positive when the number of spots was greater than three times that obtained with the negative control (control without peptides) with a minimum of 50 spots.

e) Statistical Methods

Statistical analyses were performed using online software http://marne. u707.jussieu.fr/biostatgv. Analysis of the percentage of peptide-specific T cell lines was conducted using a Student t-Test. Analysis of the peptide-specific CD4⁺ T cell precursor frequencies was conducted using the non-parametric Wilcoxon signed-rank test. Analysis of responding donors to peptides was conducted using a Fisher exact test. Finally, the CD4⁺ T cell precursor frequency was estimated using the Poisson distribution. Then mean frequency of peptide mix- and peptide-specific T cells was calculated for all the donors, including responders and non-responders.

2) Results & Analysis

One hundred and sixty (160) Survivin-specific CD4⁺ T lymphocyte lines were obtained from twelve normal donors (Table V). The twelve donors cover the HLA-DR haplotypes predominant in the Caucasian population (Table IV), namely: HLA-DR1, -DR3, -DR4, -DR7, -DR11, -DR13 and -DR15 and the corresponding second DR molecule (DRB3, DRB4 and DRB5). The peptide mixture induces specific CD4⁺ T lymphocyte lines in each donor, although the donor sampling was selected so as to include multiple HLA II haplotypes.

The specificity of the lines for the Survivin peptides was analysed by IFN-γ ELISpot assays using autologous PBMCs as antigen-presenting cells. CD4⁺ T lymphocyte lines against at least two of the three Survivin peptides were induced in each donors (Table V). The peptides S1, S2 and S3 were found to induce T cell responses in 92%, 75% and 100% of the tested donors, respectively demonstrating the high immunogenicity and promiscuity of these three Survivin derived peptides. However, the analysis of the frequency of responding donors and the percentage of positive T cell lines, showed that the peptide S2 is significantly less immunogenic than peptides S1 and S3, in a Fisher exact test and a Student t-Test respectively, whereas no significant difference in immunogenicity level was detected between peptides S1 and S3.

TABLE V
Total number of peptide-specific CD4⁺ T cell lines for each donor studied

| | | | | | | | | | | | | | Total | | |
|---|---|---|---|---|---|---|---|---|---|---|---|---|---|---|---|
| | | | | | | | | | | | | | No. | | |
| Peptides | | | | | | | | | | | | | of | % of | Frequency |
| used in | | | | | | | | | | | | | positive | positive | of |
| ELISpot | Number of peptide-specific T cell lines at ELISpot assay | | | | | | | | | | | | T cell | T cell | responding |
| assay | D1 | D2 | D3 | D4 | D5 | D6 | D7 | D8 | D9 | D10 | D11 | D12 | lines | lines | donor |
| S1 | 4 | 4 | 3 | 3 | 0 | 3 | 12 | 7 | 9 | 11 | 4 | 4 | 64 | 18 | 92 |
| S2 | 2 | 0 | 1 | 1 | 4 | 0 | 16 | 0 | 1 | 2 | 7 | 3 | 37 | 10 | 75 |
| S3 | 2 | 8 | 5 | 3 | 3 | 3 | 12 | 5 | 4 | 7 | 6 | 1 | 59 | 16 | 100 |
| Mix | 8 | 12 | 9 | 7 | 7 | 6 | 40 | 12 | 14 | 20 | 17 | 8 | 160 | 44 | 100 |

Legend of Table V

For each donor, 30 CD4⁺ T cell lines were seeded for each priming condition.

Total number (No.) and percentage (%) of the positive CD4⁺ T cell lines, for each peptide analyzed in ELISpot assays are reported on the right of the Table. Frequency of responding donors also was reported. D1 to D12=donor 1 to donor 12.

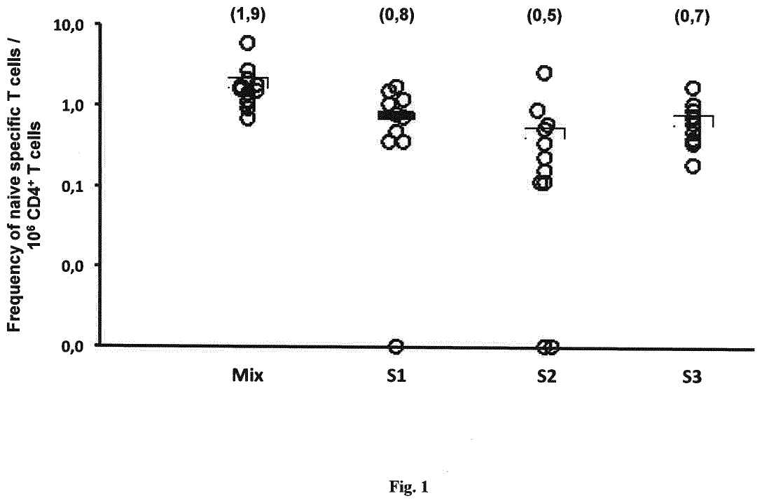

The frequency of CD4⁺ T cells precursor specific to peptides S1, S2 and S3 was evaluated for each healthy donor tested (FIG. 1). CD4+T lymphocytes obtained from the PBMCs of 12 naïve healthy donors were stimulated three times, one week apart, with autologous dendritic cells loaded with a mixture of the Survivin LSPs (S1, S2 and S3). The specificity of the obtained T lymphocyte lines was evaluated by IFN-γ ELISpot assays using autologous PBMCs in the presence or absence of the mix or the individual Survivin peptides. The mean frequency of Survivin peptide mix- and peptide-specific T cells was calculated for all the donors, including responders and non-responders. Black bars and numbers in brackets represent the mean of the frequency of all SVX-1 vaccine (Mix)- and peptide-specific CD4⁺ T cells including responding and non-responding donors.

This evaluation confirmed that peptide S2 was less immunogenic than the two others, although differences were not statistically significant. The mean frequencies of CD4⁺ T cells specific against the peptides S1, S2 and S3, varied from 0.5 to 0.8 CD4⁺ T cell per million of circulating CD4⁺ T cells T cells. The mean size of peptide mix specific-T cell repertoire was of 1.9 specific-CD4⁺ T cell per million of circulating CD4⁺ T cells, corresponding to a high level of immunogenicity.

These data predict a very high immunogenicity of the individual and the mix of inventive polypeptides in human, irrespective of the individual's HLA type. In addition, the identified relatively high size of the T cell repertoire specific to the individual and the mix (about 2 specific T-cells per Million of circulating CD4⁺ T-cells) of the inventive polypeptides, suggests that the polypeptides are potentially immunogenic in human with a T-cell repertoire not deleted in the thymus or tolerized by peripheral immunosuppressive mechanisms. It also further confirmed their potential in vaccination to generate vaccine-specific T cell responses.

### Example 2

T Cell Immunogenicity of the SVX-1 Vaccine and its Individual LSPSs in Different Mouse Strains/Models

T cell immunogenicity of the mixture and individual Survivin derived polypeptides (S1, S2 and S3) was evaluated in different mouse strains (C57BL/6 (H2^{b}); BALB/c (H2^{d}) and CBA (H2^{k})) and in the pre-clinical mouse model HLA-DRB1*0101, HLA-A*0201 (with α3 from D^{b}), H-2^{/} transgenic mice (Tg HLA-A2/DR1). The objective of this study was to select the optimal genetic background to further investigate the immunogenicity and therapeutic efficacy of the SVX-1 vaccine composes of an equimolar mixture of these 3 Survivin LSPs.

1) Materials and Methods

Six (6) to ten (10) weeks old female C57BL/6, BALB/c, and CBA mice were obtained from Charles River (Saint-Germain-Nuelles, France).

The transgenic HLA-A2/DR1 mice, previously described (Pajot et al. 2004), were bred and maintained under specific pathogen-free conditions in an animal facility. Cohort of mice were vaccinated subcutaneously (s.c.), twice at two weeks interval, with 200 μl of a mixture of S1, S2 and S3 peptides (100 μg of each polypeptides), or a peptide control, admixed with adjuvant CpG-1826 (50 μg; Invivogen) and emulsified with IFA (100 μl, Sigma-Aldrich). As positive control of immunization, C57BL/6 mice were immunized with the OVA peptide (265-280, Almac) encompassing well-defined CD4⁺ and CD8⁺ T cell epitopes. One week after the last immunization (D21), spleen cells of immunized mice were harvested, and the induction of Survivin peptides-specific T cell responses was detected ex vivo using IFN-γ ELISpot assay on the total splenocytes (2×10⁵ cells) re-stimulated overnight with the mix of peptides or the individual peptides.

b) Results

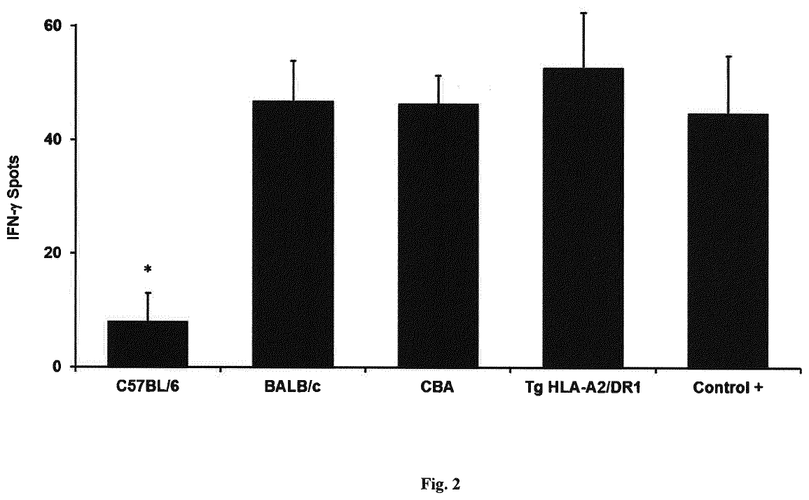

FIG. 2 illustrates the T cell immunogenicity of the mixture of the three Survivin LSPs (S1, S2 and S3; SVX-1 vaccine) in different mouse strains (C57BL/6, CBA and BALB/c) and in the pre-clinical transgenic mouse model HLA-A2/DR1. This transgenic mouse model is a relevant and unique in vivo experimental model for testing human vaccine candidates as it expresses both human HLA class I and II molecules (Johannsen et al. 2010).

Cohorts of mice were vaccinated subcutaneously (s.c.), twice at two weeks interval, with the SVX-1 vaccine, consisting of an equal mixture of each Survivin LSPs (S1+S2+S3), or a peptide control, admixed with adjuvant (CpG-1826 emulsified with IFA). As positive control of immunization (Control+), C57BL/6 mice were immunized with the OVA peptide (265-280). One week after the last immunization, the induction of SVX-1 peptides-specific T cell responses was analyzed using ex vivo IFN-γ ELISpot assays on the total splenocytes re-stimulated overnight with the mix of SVX-1 peptides. Each bar represents the mean number of spots of the duplicates±SEMs of two experiments (n=12) with *P<0.05 (Control+vs. others groups).

Immunization with the mixture of S1, S2 and S3 peptides induces strong and specific T cell responses secreting high amounts of IFN-γ in BALB/c, and CBA but not in C57BL/6 mice. These results demonstrate that the SVX-1 vaccine contains both H2^{d} and H2^{k} restricted T-cell epitopes but no H2^{b}restricted ones. SVX-1 vaccine was found to induce strong and specific T cell responses secreting high amounts of IFN-γ in Tg HLA-A2/DR1 mice.

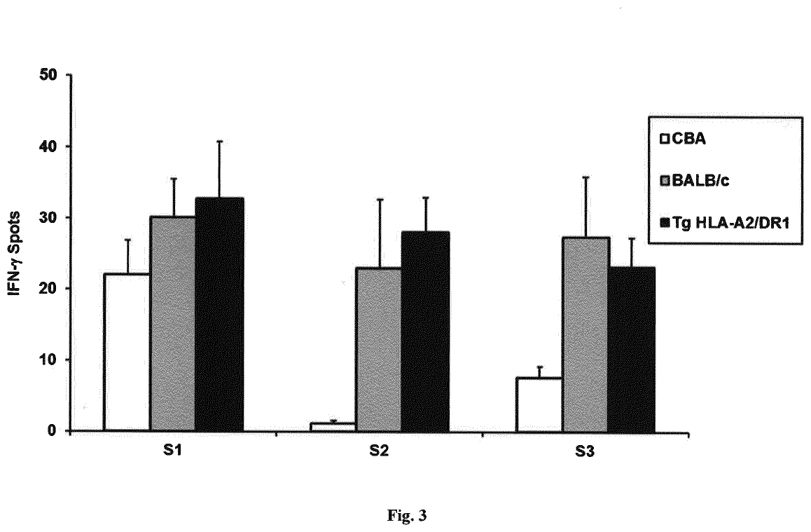

FIG. 3 illustrates the T cell immunogenicity of the three individual Survivin LSPs (17-34, 84-110 and 122-142) in different mouse strains (CBA and BALB/c) and in HLA-A2/DR1 transgenic mouse model.

Analysis of the T cell immunogenicity of the individual Survivin polypeptides revealed that they are all able to induce strong T cell responses of similar intensity in immunized BALB/c mice and in Tg HLA-A2/DR1 mice (FIG. 3).

Cohorts of mice were vaccinated subcutaneously (s.c.), twice at two weeks interval, with the SVX-1 vaccine (100 μg of S1, S2 and S3 peptides), or a peptide control (OVA 265-280), admixed with adjuvant (CpG-1826 emulsified with IFA). One week after the last immunization, the induction of SVX-1 peptides-specific T cell responses was analyzed using ex vivo IFN-γ ELISpot assays on the total splenocytes re-stimulated overnight with the individual SVX-1 peptides. Each bar represents the mean number of spots of the duplicates±SEMs of two experiments (n=6) with *P<0.05 (S1 peptide vs. others groups).

T cell responses were found to be mainly directed against the S1 peptides in immunized CBA mice. The results demonstrate that the inventive SVX-1 polypeptides all contain H2^{d} but not H2^{k} restricted T cell epitopes. The results further suggest that the S3 peptide also contains at least one HLA-DR1 and/or HLA-A2 T-cell epitope, as the S1 and S2 peptides.

### Example 3

Selection of the Optimal Adjuvant Formulation of the SVX-1 Vaccine

The T cell immunogenicity of the SVX-1 vaccine and its individual's peptides formulated with different vaccine adjuvants was compared in vivo. The aim of this study was the selection of the optimal immuno-adjuvant or adjuvant combination to formulate the SVX-1 vaccine.

1) Materials and Methods

Cohort of BALB/c mice (5 mice per group) were vaccinated s.c., twice at two weeks interval, with 200 μl of the Survivin polypeptides S1, S2 and S3 (100 μg of each peptides) formulated with different immuno-adjuvants or combinations. The vaccine adjuvants investigated are listed in the Table VI as the concentrations/quantities used in this study that were chosen according to the literature and the manufacturer's recommendations ((Valmori et al. 2007; Wick et al. 2011; Lingnau, Riedl, and von Gabain 2007; Zhou et al. 2006; Perez et al.; Garcon and Van Mechelen 2011; Speiser et al. 2005). CpG-ODN 1826 (50 μg; Invivogen) in 100 μl of incomplete Freund's adjuvant (CpG/IFA) was used as a standard adjuvant. One week after the last immunization, intensity of the T cell responses against the SVX-1 vaccine was evaluated by ex vivo IFN-γ ELISpot assays on the total splenocytes (2×10⁵ cells) re-stimulated overnight with the mixture of Survivin polypeptides.

TABLE VI
List of evaluated adjuvants

| | | | | Quantity used | |
|---|---|---|---|---|---|
| ADJUVANT | CLASS | DESCRIPTION | PROVIDER | per injection | |
| CpG-ODN | TLR 9 agonist | Synthetic oligonucleotide | Oligovax | 50 | μg |
| | | sequences | | | |
| POLY-ICLC | TLR 3 agonist | dsRNA | Oncovir.Inc | 250 | μg |
| (Hiltonol) | | | | | |
| IC31 | TLR 9 agonist | Anti-microbial peptide | Intercell | ODN1a 4 nmol | |
| | | KLK + oligonucleotide | | KLK 100 nmol | |
| | | ODN1a | | | |
| GM-CSF | — | Granulocyte macrophage | Leucomax | 20 | μg |
| | | Stimulating colony | | | |
| | | factor | | | |
| MPLA | TLR 4 agonist | Monophosphoryl lipid A | In vivogen | 20 | μg |
| IFA | Water-in-oil | Incomplete Freund's | Seppic | 100 | μl |
| | emulsion | Adjuvant | | | |
| Montanide | Water-in-oil | — | Seppic | 100 | μl |
| (ISA 51 VG) | emulsion | | | | |
| AFPL1 | TLR 4, 2 | Detergent-extracted outer | Finlay | 10 | μg |
| | and +/−9 | membrane vesicles from | Institute | | |
| | agonist | bacteria | | | |

2) Results

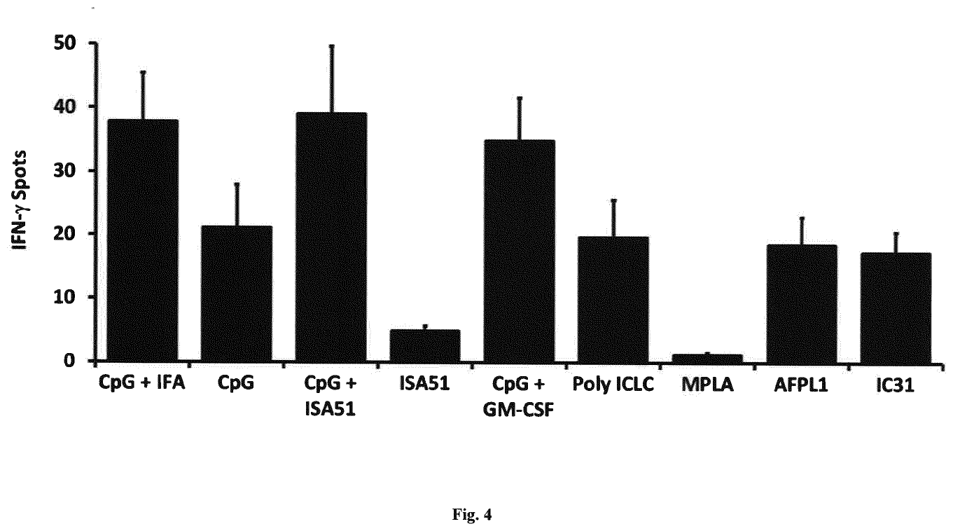

FIG. 4 illustrates the T cell immunogenicity in vivo of the SVX-1 vaccine (Peptides S1+S2+S3) formulated with various selected immuno-adjuvants. Each bar represents the mean number of IFN-γ spots of the duplicates±SEMs of two experiments (n=5) with *P<0.05 and **P<0.01, (CpG+IFA vs. other adjuvants).

Cohorts of BALB/c mice were vaccinated s.c., twice at two weeks interval, with the SVX-1 vaccine (Equal mixture of each Survivin LSPs) formulated with different immuno-adjuvants or adjuvant combinations (CpG; CpG+ISA51; ISA51; CpG+GM-CSF; Poly ICLC; MPLA; AFPL1; IC31). CpG in incomplete Freund's adjuvant (CpG+IFA) was used as a standard adjuvant. One week after the last immunization, intensity of the T cell responses against the SVX-1 vaccine was evaluated by ex vivo IFN-γ ELISpot assays on the total splenocytes re-stimulated overnight with the mix of SVX-1 peptides. Each bar represents the mean number of spots of the duplicates±SEMs of two experiments (n=5) with *P<0.05 and **P<0.01, (CpG+IFA vs. other adjuvants).

The T cell immunogenicity of the mixture of S1, S2 and S3 peptides was found to be significantly higher when formulated with IC31, CpG, AFPL1 or Poly-ICLC compared to Montanide and MPLA (FIG. 4). In addition, the combination of CpG with GM-CSF but in particular with Montanide (ISA 51 VG) significantly increased the immunogenicity of the mixture of Survivin polypeptides.

### Example 4

Pre-Clinical Proof-of-Concept Studies on the Formulated SVX-1 Vaccine

Pre-clinical proof-of-concept (PoC) studies were performed on the formulated SVX-1 vaccine to evaluate its immunogenicity and anti-tumoral activity in reliable tumour grafts animal models. The aim was to demonstrate the efficiency of the formulated SVX-1 vaccine in a context of pre-existing tumour.

1) Materials and Methods

a) Mouse Tumour Models

Three tumour cell lines in the BALB/c genetic background were selected for the tumour rejection assays. CT26 (Colorectal carcinoma), Renca (Renal Adenocarcinoma), and A20 (B cell lymphoma).

The CT26 and Renca tumour cell lines were transfected with a plasmid containing the whole human Survivin sequence (pcDNA3-hSurvivin). After several round of in vitro selection and amplification, the stability and intensity of human Survivin (hSurvivin) expression were analysed in the transfected tumour cell lines using intra-cytoplasmic staining (CT26-T and Renca-T).

The Renca and CT26 tumour cell lines were chosen regarding the high immunogenicity of the SVX-1 Survivin vaccine in BALB/c mice and as the pattern of growth of these tumour cells accurately mimics that of human adult lymphoma, and renal (RCC) and colorectal cell carcinoma, particularly with regard to spontaneous metastasis to lung and liver. Finally, the A20 tumour cell line was selected for its high expression of both mouse Survivin and MHC class II molecules that were confirmed using flow cytometry staining.

b) Evaluation of Therapeutic Efficacy of the SVX-1 Vaccine—Tumour Rejection Assays

Therapeutic efficacy of the SVX-1 vaccine was analysed using tumour rejection assays with a therapeutic setting in BALB/c mice. Cohorts of 9-10 BALB/c mice were engrafted s.c. with one of the tumour model (2×10⁵ CT26-T cells or 5×10⁵ Renca-T cells, or 2.5×10⁵ A20 cells). When tumours reached 4 to 6 mm in diameter (day 5 for CT26-T and Renca-T, and day 10 for A20), mice were immunized twice with 200 μl of the formulated SVX-1 vaccine (100 μg of each LSP) one week apart.

Additional control groups were added: (a) Mice engrafted with transfected cell line without vaccination; (b) Mice vaccinated but not engrafted. Tumour size was monitored every other day. Several days post tumor challenge (PTC) (D26 and 36 in experiments with CT26-T cells, D36 in experiments with A20 cells, and D28 in experiments with Renca-T cells), the intensity of the SVX-1 specific T cell responses was evaluated using IFN-γ ELISpot assays on total splenocytes restimulated overnight with the mix of SVX-1 peptides.

2) Results

a) SVX-1 Therapeutic Efficacy Against Established Colorectal Tumour Cells Expressing the Human Survivin (CT26-T)

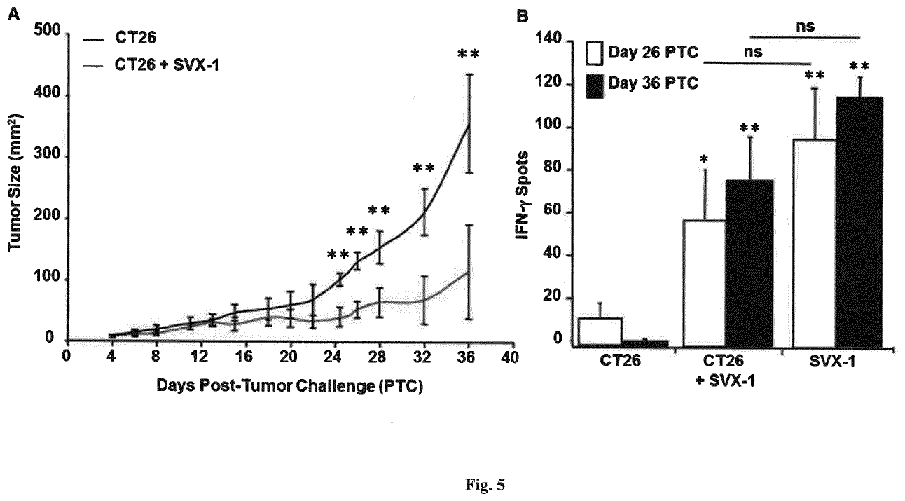

FIG. 5 illustrates the therapeutic efficacy of SVX-1 against established colorectal tumour cells expressing the human Survivin (CT26-T). FIG. 5A: Follow-up of tumor size in cohorts of BALB/c mice engrafted s.c. with CT26-T tumor cells (day 0) and subsequently immunized twice (days 5 and 12) with the SVX-1 vaccine (100g of each LSP) at one-week interval (CT26-T+SVX-1). Mice engrafted with tumor cells without vaccination were used as control group (CT26-T). Each dot represents the mean of tumor size monitored every other day±SEMs of one experiment (n=9) with *P <0.05 and **P<0.01, (Group CT26-T+SVX-1 vs. control group). FIG. 5B: Analysis of the intensity of SVX-1-specific T-cell responses, on days 26 and 36 post tumor challenge, by IFN-γ ELISpot assays on total splenocytes after an overnight in vitro restimulation with the pool of SVX-1 peptides. Each bar represents the mean number of spots of the duplicates±SEMs of one experiment (n=5) with *P<0.05 and **P<0.01, (CT26-T vs. other groups). ns: not significant.

The growth of established CT26-T tumour cells was found to be significantly impaired by day 24 PTC in the group of mice immunized with the formulated SVX-1 vaccine (FIG. 5A, lower trace) compared to the control group (FIG. 5A, upper trace). The difference was found to be more and more significant over time (FIG. 5A). Analysis of the induction of T cell responses in the different groups of mice at day 26 PTC demonstrated that the SVX-1 vaccine induced intense SVX-1-specific T cell responses secreting high amounts of IFN-γ (FIG. 5B) in all the SVX-1 immunized group of mice compared to the control group. In addition, the intensity of the SVX-1-specific T cell responses was not significant impaired in presence of the CT26-T tumor cells but was even slightly improved at day 36 PTC in all the SVX-1 immunized groups.

b) SVX-1 Therapeutic Efficacy Against Established Renal Cancer Model (Renca-T)

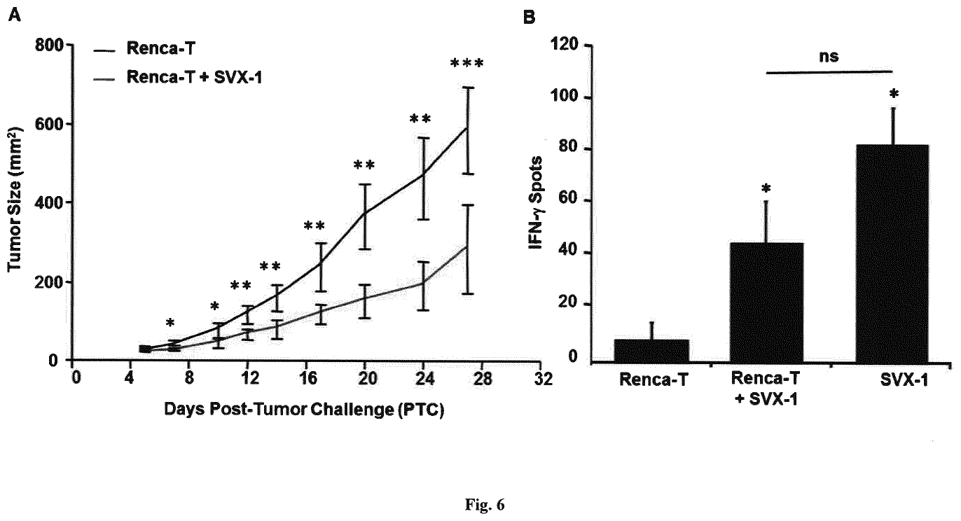

FIG. 6 illustrates therapeutic efficacy of SVX-1 against established Renal cancer model expressing the human Survivin (Renca-T). FIG. 6A: Follow-up of tumor size in cohorts of BALB/c mice engrafted s.c. with Renca-T tumor cells (day 0) and subsequently (day 5) immunized twice with the SVX-1 vaccine (100 μg of each LSP) at one-week interval (Renca-T+SVX-1). Mice engrafted with tumor cells without vaccination were used as control group (Renca). Each dot represents the mean of tumor size monitored every other day±SEMs of one experiment (n=9) with *P<0.05, **P<0.01, and ***P<0.001 (Group Renca-T+SVX-1 vs. control group). FIG. 6B: Analysis of the intensity of SVX-1-specific T-cell responses, on day 28 post tumor challenge, by IFN-γ ELISpot assays on total splenocytes after an overnight in vitro restimulation with the mix of SVX-1 peptides. Each bar represents the mean number of spots of the duplicates±SEMs of one experiment (n=9) with *P<0.05 (Renca-T vs. other groups). ns: not significant.

Similarly to what observed in the CT26-T engrafted mice, the growth of the Renca tumor cells was found to be significantly impaired by day 7 PTC in the group of Renca-T engrafted mice immunized with the SVX-1 vaccine (FIG. 6A, lower trace) compare to the control group (FIG. 6A, upper trace). This difference was also found to be more and more significant over time (FIG. 6A). Analysis of the induction of T cell responses in the different groups of mice at day 28 PTC also demonstrated that the SVX-1 vaccine induced intense SVX-1 specific T cell responses secreting high amounts of IFN-γ in all the SVX-1 immunized groups of mice compared to the control group. In addition, SVX-1-specific T cell responses was found to be slightly impaired in presence of the Renca-T tumor cells but the difference was not significant in a student t-Test (FIG. 6B).

c) SVX-1 Therapeutic Efficacy Against Established B Cell Lymphoma (A20)

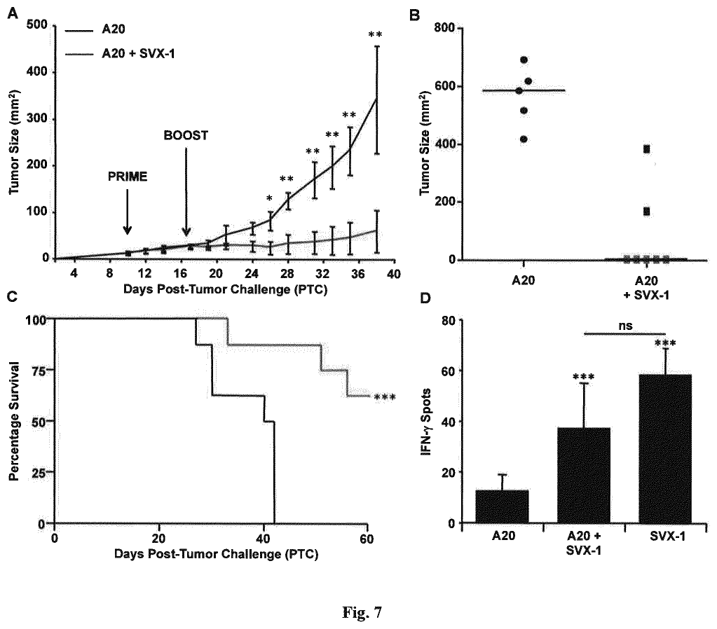

FIG. 7 illustrates the therapeutic efficacy of the SVX-1 vaccine against an established B cell lymphoma model (A20) and the induction of long-term survival. FIG. 7A: Follow-up of tumor size in cohorts of BALB/c mice engrafted s.c. with A20 tumor cells (day 0) and subsequently (day 10) immunized twice with the SVX-1 vaccine (100 μg of each LSP) at one-week interval (A20+SVX-1). Mice engrafted with tumor cells without vaccination were used as control group (A20). Each dot represents the mean of tumor size monitored every other day±SEMs of one experiment (n=8) with *P<0.05 and **P<0.01, (Group A20+SVX-1 vs. control group). FIG. 7B: Tumor sizes (mm²) in the different groups of mice at day 52 post tumor challenge. Each dot represents a single mouse. Squares located on the x-axis for the A20+SVX-1 group represent mice that were able to completely eradicate the A20 tumors. FIG. 7C illustrates mice were monitored for survival for 60 day-period post-tumor challenge. Mice that became moribund due to tumor burden were killed. Survival was plotted according to Kaplan-Meier methods (***P<0.001). FIG. 7D: Intensity of SVX-1 specific T-cell responses in the different groups of mice.

Evaluation was performed on day 36 post tumor challenge using IFN-γ ELISpot assays on total splenocytes restimulated one week in vitro with the pool of SVX-1 peptides. Mice only immunized with the SVX-1 vaccine were used as positive control (SVX-1). Data are presented as means of IFN-γ spots±S.D. in the different groups of mice (3 mice per groups) with ***P<0.001 (A20 vs. Other groups). ns: not significant.

Treatments with SVX-1 vaccine (FIG. 7A, lower trace) significantly suppress the growth of established A20 cells as compared to control group (FIG. 7A, upper trace). In addition, treatment with the SVX-1 vaccine was found to completely eradicate A20 tumors in 71% of the treated mice (5/7), 52 days post-tumor challenge (FIG. 7B), and to induce long-term survival as 60% (6/10) of the SVX-1 treated animals survived over a 60-days period (FIG. 7C, upper trace) whereas 100% (10/10) of the untreated animals were moribund by day 42 (FIG. 7C, lower trace).

This was also associated with the induction of strong SVX-1 specific T cell responses secreting high amounts of IFN-γ as observed in the different groups of mice at day 36 (FIG. 7B).

3) Analysis

Results of the pre-clinical PoC studies clearly demonstrated the high therapeutic efficacy of the SVX-1 vaccine against various established tumour models in mice, such as colorectal carcinoma, and renal adenocarcinoma models expressing the human Survivin. In addition, the high therapeutic efficacy of the SVX-1 was found to be associated with the induction of intense and long-lasting SVX-1 specific T-cell responses not impaired by the presence of the tumour cells.

Finally, the results also highlighted the high therapeutic efficacy of the SVX-1 vaccine in suppressing the growth of A20 tumour cells expressing the mouse Survivin, without any sign of toxicity in mice in a period of 50 days. This demonstrates the capacity of the SVX-1 induced T cell lines to cross-react with mouse survivin derived T cell epitopes presented by the A20 tumour cells.

All these data represent relevant pre-clinical proof of concepts of the high therapeutic efficacy and safety of the SVX-1 vaccine and support the used of the inventive polypeptides to treat and prevent tumour growth.

### Example 5

Evaluation of the Capacity of the Formulated SVX-1 Vaccine to Induce Anti-Tumor Memory Responses

An effective therapeutic cancer vaccine may induce potent anti-tumor immune responses able to eradicate the tumors but also anti-tumor memory responses for long-lasting protection against relapses. The capacity of the SVX-1 vaccine to induce such memory responses was thus evaluated by rechallenging SVX-1 treated mice, which eradicated A20 tumors in primary responses, with live A20 cells.

1) Materials and Methods

BALB/c mice (n=5) who completely eradicated A20 tumors in primary response (FIG. 7B) were rechallenged with live A20 cells (2.5×10⁵ cells) 60 days after primary inoculation (Tumor rechallenge).

Age-matched naïve BALB/c mice (n=5) were used as control (A20 naïve mice). Tumor sizes were assessed for 36 days post-tumor (re)challenge. Mice were monitored for survival for 60 day-period post-tumor (re)challenge.

2) Results and Analysis

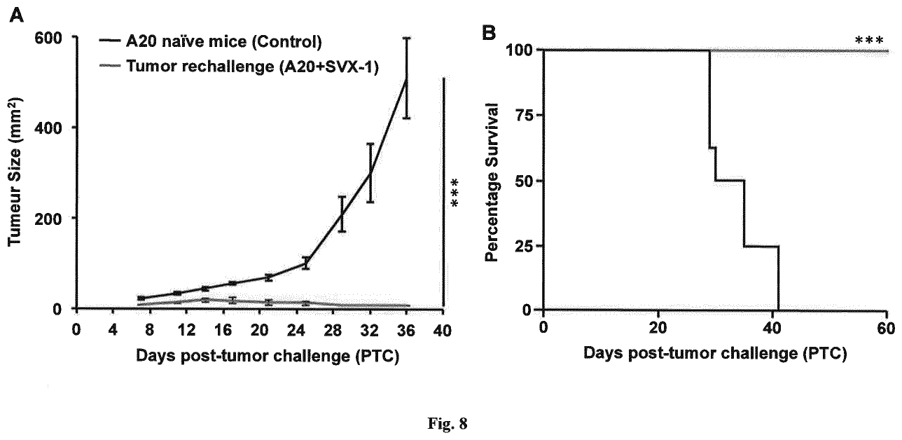

FIG. 8 illustrates the capacity of the SVX-1 vaccine to induce effective anti-tumor memory responses against B lymphoma tumor cells (A20). FIG. 8A: BALB/c mice (n=5) who completely eradicated A20 tumors in primary response (FIG. 5, Panel B) were rechallenged with live A20 cells 60 days after primary inoculation (Tumor rechallenge; lower trace). Age-matched naïve mice (n=5) were used as control (A20 naïve mice; upper trace). Tumor sizes were assessed for 36 days post-tumor (re)challenge. Data are presented as means tumor size (mm2)±S.D. in the different groups of mice, with ***P<0.001. FIG. 8B: Mice were monitored for survival for 60 day-period post-tumor (re)challenge. Mice that became moribund due to tumor burden were killed. Survival was plotted according to Kaplan-Meier methods (***P<0.001). Upper trace represents Tumor rechallenge group, and lower trace represents A20 naïve mice group.

All vaccinated mice were resistant to secondary A20 rechallenge even 60 days after primary challenge (FIG. 8A) with 100% survival over a 60-days period (FIG. 8B), demonstrating the capacity of the SVX-1 vaccine to induce effective anti-tumor memory responses against B lymphoma tumor cells.

### Example 6

Evaluation of the Capacity of the Formulated SVX-1 Vaccine to Induce Anti-Tumoral CD4⁺ and CD8⁺ T Cell Responses

Therapeutic efficacy of SVX-1 vaccine against established MCH class I+/II⁻ (CT26) and MHC class I⁺/II⁺ (A20) tumour models was evaluated in CD8⁺-depleted mice. The aims of this study were 1) to evaluate the capacity of the SVX-1 vaccine to induce anti-tumoral CD8+ and CD4⁺ T cell responses, and 2) to determine their role in SVX-1 therapeutic efficacy against established tumours.

1) Materials and Methods

Cohorts of 8 BALB/c mice were engrafted subcutaneously with A20 or CT26-T tumour cells (2.5×10⁵ cells) in the abdominal flank. When tumours reach 5-10 mm² in diameter (day 5 for CT26-T and day 7 for A20), mice were immunized with the formulated SVX-1 vaccine (100g of each LSP) and then boosted 7 days later (Tumour+SVX-1). Groups of mice were depleted of CD8⁺ cells using 100 g of anti-CD8 antibodies injected intraperitoneally (i.p.) the day before each SVX-1 immunization. The efficacy of the CD8⁺ cell depletion was confirmed by flow cytometry staining, using anti-CD4 and anti-CD8 antibodies, in the spleen of a group of mice treated with the depleting antibody. Additional control groups were added: (a) Mice engrafted with tumour cells but not immunized (Tumour); (b) Mice immunized but not grafted with tumour cells (SVX-1). Tumour size was monitored every other day and the induction of SVX-1 specific T-cell responses was evaluated several days post tumour challenge (D32 in experiments with CT26-T cells and D32 in experiments with A20 cells), in IFN-γ ELISpot assays on total splenocytes restimulated overnight with the mix of SVX-1 peptides.

2) Results and Analysis

a) Efficacy of the CD8⁺ Cells Depletion

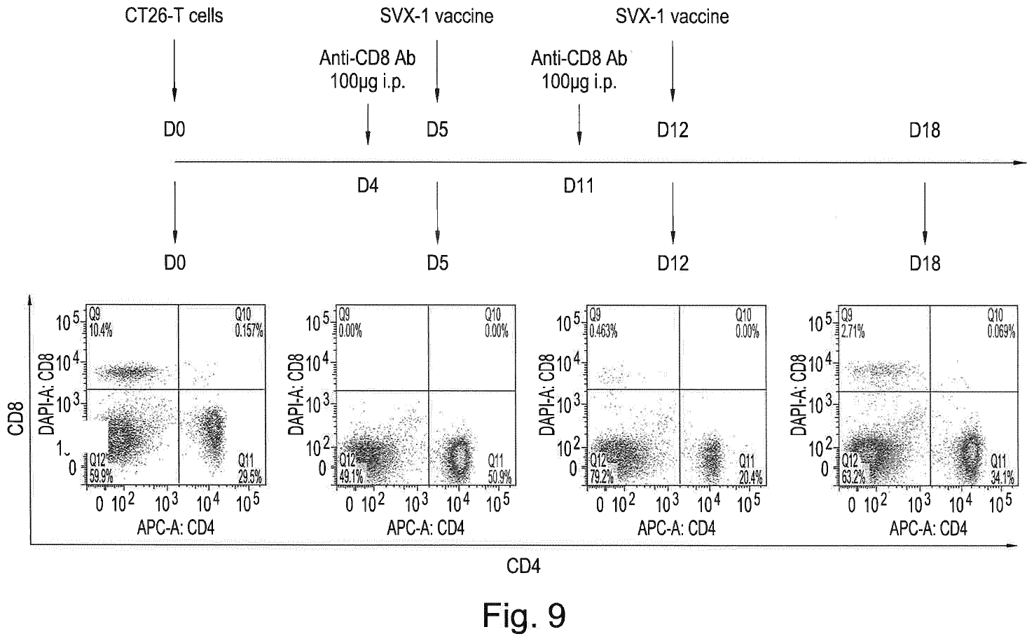

FIG. 9 illustrates the percentage of CD8⁺ cells in the spleen of BALB/c mice before and one day after treatment with an anti-CD8 depleting antibody (at days 5 and 12) by flow cytometry staining, using anti-CD4 and anti-CD8 antibodies.

The percentage of CD8+ cells was evaluated in the spleen of BALB/c mice before and one day after each treatment with an anti-CD8 depleting antibody (days 5 and 12) using by flow cytometry staining, using anti-CD4 and anti-CD8 antibodies. While 10% of CD8⁺ cells are detected in the spleen of untreated mice, 0% and 0.463% of CD8+ cells are detected one day post anti-CD8 antibody treatment (FIG. 9). These results demonstrated that the treatment of mice with the anti-CD8 depleting antibody significantly deplete the CD8⁺ cells. On day 18, only 2.71% of CD8⁺ cells are detected in the spleen of treated mice demonstrating that the pool of CD8⁺ cells are not fully restore in mice one week after the last treatment.

b) Therapeutic Efficacy of the SVX-1 Vaccine Against Established Tumour Cells, in CD8-Depleted Mice

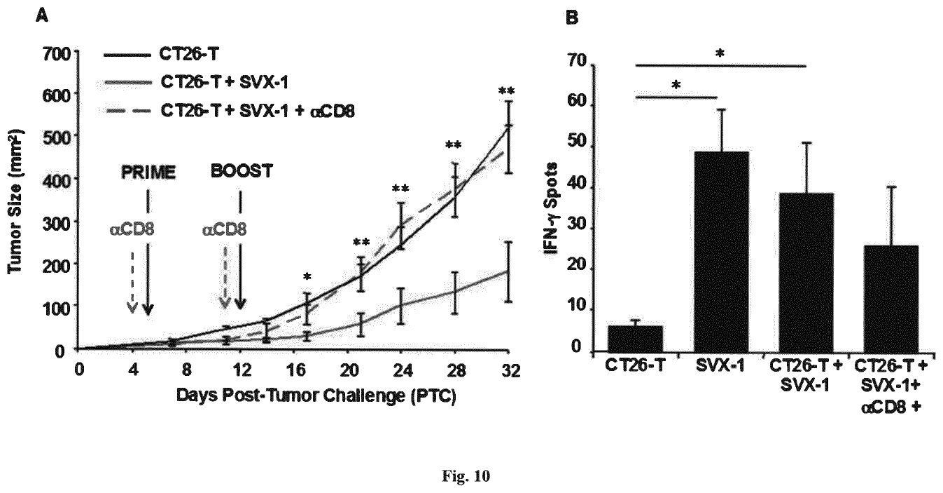

FIG. 10 illustrates the therapeutic efficacy of the SVX-1 vaccine against established MHC class I⁺/II⁻ colorectal tumour cells (CT26) in CD8-depleted mice. FIG. 10A: Follow-up of tumor size in cohorts of CT26 tumor-bearing BALB/c mice depleted (upper dashed trace) or not (lower solid trace) of CD8⁺ cells (Days 4 and 11, see dashed arrows) and immunized with the formulated SVX-1 therapeutic cancer vaccine (100g of each LSP on days 7 and 14 PTC; See solid arrows). Untreated mice were used as control (CT26-T, upper solid trace). Data are presented as means tumor size (mm²)±S.D. in the different groups of mice, with *P<0.05 and **P<0.01. FIG. 10B: Intensity of SVX-1 specific T-cell responses in the different cohorts of CT26 engrafted mice. Evaluation was performed on day 32 post tumor challenge using IFN-γ ELISpot assays on total splenocytes restimulated with the pool of SVX-1 peptides. Mice only immunized with the SVX-1 vaccine were used as positive control (SVX-1). Data represent the mean and standard error of 8 mice per group with *P<0.05.

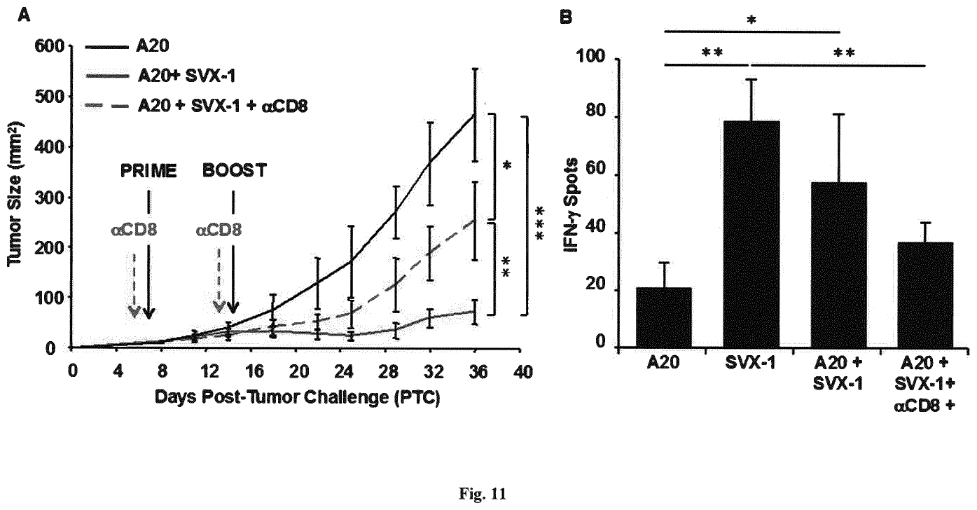

FIG. 11 illustrates the therapeutic efficacy of the SVX-1 vaccine against MHC class I⁺/II⁺ tumour cells (A20) in CD8-depleted mice. FIG. 11A: Follow-up of tumor size in cohorts of A20 tumor-bearing BALB/c mice depleted (middle dashed trace) or not (lower solid trace) of CD8⁺ cells (Days 6 and 13, see dashed arrows) and immunized with the adjuvanted SVX-1 therapeutic cancer vaccine (100 μg of each LSP on days 7 and 14 PTC; See solid arrows). Untreated mice (A20) were used as control (upper solid trace). Data are presented as means tumor size (mm²) ±S.D. in the different groups of mice, with *P<0.05 and **P<0.01. FIG. 11B: Intensity of SVX-1 specific T-cell responses in the different cohorts of CT26 engrafted mice. Evaluation was performed on day 32 post tumor challenge using IFN-γ ELISpot assays on total splenocytes restimulated with the pool of SVX-1 peptides. Mice only immunized with the SVX-1 vaccine were used as positive control (SVX-1). Data represent the mean and standard error of 8 mice per group with *P<0.05.

In CD8-depleted mice, the therapeutic efficacy of the SVX-1 vaccine was totally abolished against established MHC class I⁺/II⁻ colorectal tumour cells (CT26) (FIG. 10A) whereas it was only significantly impaired against MHC class I⁺/II⁺ tumour cells (A20) (FIG. 11A). In addition, induction of SVX-1 specific T-cell responses was slightly decreased in CD8-depleted mice compared to control mice, but the differences were not statistically significant in a student t-Test (FIGS. 10B and 11B).

These results highlighted the capacity of the SVX-1 vaccine to induce strong anti-tumoral CD8+ T-cell responses and demonstrated their crucial role in the therapeutic efficacy of the SVX-1 vaccine against established MHC class I tumour cells. In addition, results suggested that the SVX-1 vaccine is also able to induce anti-tumoral CD4⁺ T-cell responses playing a direct role in the therapeutic efficacy of the SVX-1 vaccine against established MHC class II⁺ tumour cells.

### Example 7

Evaluation of Spontaneous Basal SVX-1-Specific T Cell Responses in Cancer Patients

The objective of this study was to monitor the frequency and intensity of T cell precursors specific to the SVX-1 vaccine and its individual peptides circulating in cancer patients.

1) Materials and Methods

a) Blood Samples from Cancer Patients

Peripheral blood from 7 lung cancer patients was used to monitor the presence of SVX-1 peptides specific T cells. The cancer patients were recruited at the Hôpital Europeen Georges Pompidou (Paris, France). This study was conducted in accordance with French laws and after approval by the local ethics committee. Blood cells were also collected from 3 anonymous healthy donors at the Etablissement Français du Sang (EFS, Rungis, France) as buffy-coat preparations after informed consent and following EFS guidelines. The blood from the healthy donors served as negative controls.

b) Assessment of SVX-1-Specific T Cell Response

PBMC were isolated by density centrifugation on Ficoll-Hyperpaque gradients (Sigma-Aldrich). PBMC were cultured for 6 days at 2×10⁶ cells/ml in 2 ml per well with complete RPMI medium supplemented with 10% FCS. In each well, a pool of SVX-1 peptides was added at a concentration of 10 μg/ml. On day 2 after the beginning of the culture, IL-2 (Chiron) was added at 20 IU/ml in standard conditions. After 6 days of culture, ELISpot assays were performed using PHA-activated cells pulsed with the pool of SVX-1 peptides or the individual peptides (S1, S2 or S3) as antigen presenting cells (APCs). Briefly, PHA-activated cells were obtained by a culture of autologous PBMC in RPMI 1640 medium containing 10% FCS and supplemented with 10 μg/ml PHA-P (Sigma-Aldrich).

At day 3, IL-2 (20 IU/ml) and IL-7 (10 ng/ml) were added to the culture. At day 6, PHA-activated cells were fixed with 1% PFA for 30 min at 4° C., washed three times with PBS, and pulsed for 2 h at 37° C. with the various peptides at 10 μg/ml in serum-free medium (AIM V medium). Ninety-six-well polyvinylidene difluoride plates (Millipore) were coated with 100 μl capture anti-human IFN-y mAb (Diaclone) and incubated overnight at 4° C. The plates were then saturated with 2% skimmed milk and incubated for 2 h at room temperature. Effector cells (10⁵) and PHA-activated T cells (5×10⁴) pulsed with the peptides were added to triplicate wells at 10⁵ cells/well in AIM V medium for 20 h at 37° C. in 5% CO2. At the end of incubation, cells were washed and the second biotinylated anti-IFN-γ mAb (Diaclone) was added to the plate for 90 min at 37° C., followed by streptavidin-alkaline phosphatase conjugate (Diaclone) for 1 h at 37° C. and by NBT/5-bromo-4-chloro-3-indolylphosphate toluidine mix (Diaclone) as substrate.

Spots were counted using an automated stereomicro-scope (Zeiss). The number of specific T cells expressed as spot-forming cells/10⁵ cells was calculated after subtracting negative control values (background). Cells incubated with medium alone or PMA (100 ng/ml) (Sigma-Aldrich) and ionomycin (10 μM) (Sigma-Aldrich) were used as negative and positive controls, respectively.

2) Results and Analysis

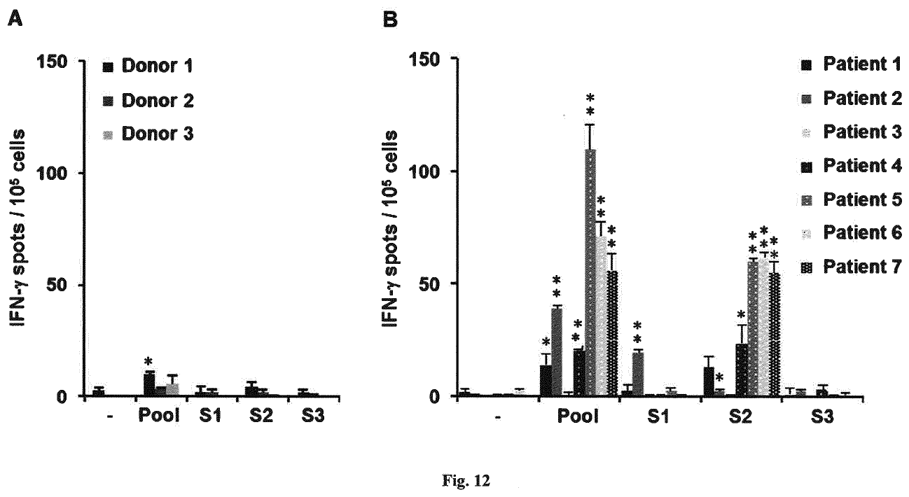

FIG. 12 illustrates spontaneous T-cell responses against SVX-1 peptides in the blood of healthy donors (A) and lung cancer patients (B). PBMCs from 7 lung cancer patients and 3 healthy donors was screened for spontaneous T-cell reactivity against the mix (S1+S2+S3) and individual SVX-1 peptides, in IFN-γ ELISpot assays, after one week of in vitro restimulation with the pool of SVX-1 peptides. Data are the mean±SEMs of one experiment in triplicate with *P<0.05 and **P<0.03-Medium vs. Pool or Individual peptides

Spontaneous SVX-1 specific T cell responses were detected in the blood of 6/7 lung cancer patients (FIG. 12B) but none in the blood of the healthy control donors (FIG. 12A). T cell responses were found to be mainly against the S2 peptide (5/6 positive patients), although S1 peptide specific T-cell responses were detected, supporting its immunogenicity.

These results demonstrated that SVX-1-specific T-cell repertoire is spontaneously stimulated in lung cancer patients, but not in healthy donors, indicating the absence of immune tolerance against the SVX-1 vaccine in such patients. These results further suggest that the SVX-1 vaccine could potentially boost the activation of such specific precursors in lung cancer patients. This also underlined the universal nature of the promiscuous HLA-DR-restricted SVX-1 peptides.

### REFERENCES

Adamkov, M, K Kajo, D Vybohova, J Krajcovic, F Stuller, and J Rajcani. 2012. “Correlations of Survivin Expression with Clinicomorphological Parameters and Hormonal Receptor Status in Breast Ductal Carcinoma.” Neoplasma 59 (1): 30-37. http://www.ncbi.nlm.nih.gov/pubmed/22103896.
Adida, C, C Haioun, P Gaulard, E Lepage, P Morel, J Briere, H Dombret, et al. 2000. “Prognostic Significance of Survivin Expression in Diffuse Large B-Cell Lymphomas.” Blood 96 (5): 1921-25. http://www.ncbi.nlm.nih.gov/pubmed/10961895.
Ahmed, Manal Basyouni, Hanan Hussien Shehata, Mona Moussa, and Tamer Mohamed Ibrahim. 2012. “Prognostic Significance of Survivin and Tumor Necrosis Factor-Alpha in Adult Acute Lymphoblastic Leukemia.” Clinical Biochemistry 45 (1-2): 112-16. doi:10.1016/j.clinbiochem.2011.08.1147.
Altieri, Dario C. 2003. “Survivin, Versatile Modulation of Cell Division and Apoptosis in Cancer.” Oncogene 22 (53): 8581-89. doi:10.1038/sj.onc.1207113.
Andersen, M H, L O Pedersen, J C Becker, and P T Straten. 2001. “Identification of a Cytotoxic T Lymphocyte Response to the Apoptosis Inhibitor Protein Survivin in Cancer Patients.” Cancer Research 61 (3): 869-72. http://www.ncbi.nlm.nih.gov/pubmed/11221872.
Andersen, M H, R B Soerensen, J C Becker, and P thor Straten. 2006. “HLA-A24 and Survivin: Possibilities in Therapeutic Vaccination against Cancer.” J Transl Med 4: 38. doi:10.1186/1479-5876-4-38.
Andersen, M H, I M Svane, J C Becker, and P T Straten. 2007. “The Universal Character of the Tumor-Associated Antigen Survivin.” Clin Cancer Res 13 (20): 5991-94. doi:10.1158/1078-0432.CCR-07-0686.
Audran, Régine, Floriana Lurati-Ruiz, Blaise Genton, Hildur E Blythman, Opokua Ofori-Anyinam, Christophe Reymond, Giampietro Corradin, and Francois Spertini. 2009. “The Synthetic Plasmodium Falciparum Circumsporozoite Peptide PfCS102 as a Malaria Vaccine Candidate: A Randomized Controlled Phase I Trial.” PloS One 4 (10): e7304. doi:10.1371/journal.pone.0007304.
Barany, G, N Kneib-Cordonier, and D G Mullen. 1987. “Solid-Phase Peptide Synthesis: A Silver Anniversary Report.” International Journal of Peptide and Protein Research 30 (6): 705-39. http://www.ncbi.nlm.nih.gov/pubmed/3326854.
Baytekin, Firat, Burcin Tuna, Ugur Mungan, Guven Aslan, and Kutsal Yorukoglu. 2011. “Significance of P-Glycoprotein, p53, and Survivin Expression in Renal Cell Carcinoma.” Urologic Oncology: Seminars and Original Investigations 29 (5). Elsevier Inc.: 502-7. doi:10.1016/j.urolonc.2009.09.001.
Becker, J C, M H Andersen, V Hofmeister-Muller, M Wobser, L Frey, C Sandig, S Walter, et al. 2012. “Survivin-Specific T-Cell Reactivity Correlates with Tumor Response and Patient Survival: A Phase-II Peptide Vaccination Trial in Metastatic Melanoma.” Cancer Immunol Immunother 61 (11): 2091-2103. doi:10.1007/s00262-012-1266-9.
Berntsen, Annika, Redas Trepiakas, Lynn Wenandy, Poul F Geertsen, Per thor Straten, Mads H Andersen, Anders E Pedersen, et al. 2008. “Therapeutic Dendritic Cell Vaccination of Patients with Metastatic Renal Cell Carcinoma: A Clinical Phase 1/2 Trial.” Journal of Immunotherapy (Hagerstown, Md.: 1997) 31 (8): 771-80. doi:10.1097/CJI.0b013e3181833818.
Bird, A P, M H Taggart, R D Nicholls, and D R Higgs. 1987. “Non-Methylated CpG-Rich Islands at the Human Alpha-Globin Locus: Implications for Evolution of the Alpha-Globin Pseudogene.” The EMBO Journal 6 (4): 999-1004. http://www.pubmedcentral.nih.gov/articlerender.fcgi?artid=553495&tool=pmcentrez&render type=abstract.
Birr, Christian. 1985. “Principles of Peptide Synthesis. VonM. Bodanszky. Springer-Verlag, Berlin 1984. XVI, 308 S., Geb. DM 148.00.—ISBN 3-540-12395-4.” Angewandte Chemie 97 (4): 360-61. doi:10.1002/ange.19850970445.
Boidot, Romain, Frédérique Vegran, and Sarab Lizard-Nacol. 2009. “Predictive Value of Survivin Alternative Transcript Expression in Locally Advanced Breast Cancer Patients Treated with Neoadjuvant Chemotherapy.” International Journal of Molecular Medicine 23 (2): 285-91. http://www.ncbi.nlm.nih.gov/pubmed/19148555.
Carpentier, Antoine F, Gregor Auf, and Jean-Yves Delattre. 2003. “CpG-Oligonucleotides for Cancer Immunotherapy: Review of the Literature and Potential Applications in Malignant Glioma.” Frontiers in Bioscience: A Journal and Virtual Library 8 (January): e115-27. http://www.ncbi.nlm.nih.gov/pubmed/12456326.
Casati, C, P Dalerba, L Rivoltini, G Gallino, P Deho, F Rini, F Belli, et al. 2003. “The Apoptosis Inhibitor Protein Survivin Induces Tumor-Specific CD8+ and CD4+ T Cells in Colorectal Cancer Patients.” Cancer Res 63 (15): 4507-15. http://www.ncbi.nlm.nih.gov/pubmed/12907624.
Charalambous, A, M Oks, G Nchinda, S Yamazaki, and R M Steinman. 2006. “Dendritic Cell Targeting of Survivin Protein in a Xenogeneic Form Elicits Strong CD4+ T Cell Immunity to Mouse Survivin.” J Immunol 177 (12): 8410-21. http://www.ncbi.nlm.nih.gov/pubmed/17142738.
Church, Sarah E., Shawn M. Jensen, Paul A. Antony, Nicholas P. Restifo, and Bernard A. Fox. 2014. “Tumor-Specific CD4+ T Cells Maintain Effector and Memory Tumor-Specific CD8+ T Cells.” European Journal of Immunology 44 (1): 69-79. doi:10.1002/eji.201343718.
Ciesielski, M J, D Kozbor, C A Castanaro, T A Barone, and R A Fenstermaker. 2008. “Therapeutic Effect of a T Helper Cell Supported CTL Response Induced by a Survivin Peptide Vaccine against Murine Cerebral Glioma.” Cancer Immunol Immunother 57 (12): 1827-35. doi:10.1007/s00262-008-0510-9.
Coffman, Robert L, Alan Sher, and Robert A Seder. 2010. “Vaccine Adjuvants: Putting Innate Immunity to Work.” Immunity 33 (4): 492-503. doi:10.1016/j.immuni.2010.10.002.
de Vos van Steenwijk, P J, M I van Poelgeest, T H Ramwadhdoebe, M J Lowik, D M Berends-van der Meer, C E van der Minne, N M Loof, et al. 2014. “The Long-Term Immune Response after HPV16 Peptide Vaccination in Women with Low-Grade Pre-Malignant Disorders of the Uterine Cervix: A Placebo-Controlled Phase II Study.” Cancer Immunol Immunother 63 (2): 147-60. doi:10.1007/s00262-013-1499-2.
Ellebaek, E, L Engell-Noerregaard, T Z Iversen, T M Froesig, S Munir, S R Hadrup, M H Andersen, and I M Svane. 2012. “Metastatic Melanoma Patients Treated with Dendritic Cell Vaccination, Interleukin-2 and Metronomic Cyclophosphamide: Results from a Phase II Trial.” Cancer Immunol Immunother 61 (10): 1791-1804. doi:10.1007/s00262-012-1242-4.
Fortugno, Paola, Nathan R Wall, Alessandra Giodini, Daniel S O'Connor, Janet Plescia, Karen M Padgett, Simona Tognin, Pier Carlo Marchisio, and Dario C Altieri. 2002. “Survivin Exists in Immunochemically Distinct Subcellular Pools and Is Involved in Spindle Microtubule Function.” Journal of Cell Science 115 (Pt 3): 575-85. http://www.ncbi.nlm.nih.gov/pubmed/11861764.
Fourcade, Julien, Pavol Kudela, Pedro A Andrade Filho, Bratislav Janjic, Stephanie R Land, Cindy Sander, Arthur Krieg, et al. 2008. “Immunization with Analog Peptide in Combination with CpG and Montanide Expands Tumor Antigen-Specific CD8+ T Cells in Melanoma Patients.” Journal of Immunotherapy (Hagerstown, Md.: 1997) 31 (8): 781-91. doi:10.1097/CJI.0b013e318183af0b.
Fukuda, Seiji, and Louis M Pelus. “Elevation of Survivin Levels by Hematopoietic Growth Factors Occurs in Quiescent CD34+ Hematopoietic Stem and Progenitor Cells before Cell Cycle Entry.” Cell Cycle (Georgetown, Tex.) 1 (5): 322-26. http://www.ncbi.nlm.nih.gov/pubmed/12461294.
- - - . 2006. “Survivin, a Cancer Target with an Emerging Role in Normal Adult Tissues.” Molecular Cancer Therapeutics 5 (5): 1087-98. doi:10.1158/1535-7163.MCT-05-0375.
Gao, J-H, F-H Chen, L Wang, H Wei, and S-L Meng. 2015. “YM155 Inhibits Tumor Growth and Enhances Chemosensitivity to Cisplatin in Osteosarcoma.” European Review for Medical and Pharmacological Sciences 19 (11): 2062-69. http://www.ncbi.nlm.nih.gov/pubmed/26125270.
Garcon, N, and M Van Mechelen. 2011. “Recent Clinical Experience with Vaccines Using MPL- and QS-21-Containing Adjuvant Systems.” Expert Rev Vaccines 10 (4): 471-86. doi:10.1586/erv.11.29.
Groner, Bernd, and Astrid Weiss. 2014. “Targeting Survivin in Cancer: Novel Drug Development Approaches.” BioDrugs 28 (1): 27-39. doi:10.1007/s40259-013-0058-x.
Haabeth, Ole Audun Werner, Anders Aune Tveita, Marte Fauskanger, Fredrik Schjesvold, Kristina Berg Lorvik, Peter O. Hofgaard, Hilde Omholt, et al. 2014. “How Do CD4+ T Cells Detect and Eliminate Tumor Cells That Either Lack or Express MHC Class II Molecules?” Frontiers in Immunology 5 (APR): 1-13. doi:10.3389/fimmu.2014.00174.
Hirohashi, Yoshihiko, Toshihiko Torigoe, Akiko Maeda, Yuki Nabeta, Kenjiro Kamiguchi, Takashi Sato, Junichi Yoda, et al. 2002. “An HLA-A24-Restricted Cytotoxic T Lymphocyte Epitope of a Tumor-Associated Protein, Survivin.” Clinical Cancer Research: An Official Journal of the American Association for Cancer Research 8 (6): 1731-39. http://www.ncbi.nlm.nih.gov/pubmed/12060610.
Hobo, W, L Strobbe, F Maas, H Fredrix, A Greupink-Draaisma, B Esendam, T de Witte, et al. 2013. “Immunogenicity of Dendritic Cells Pulsed with MAGE3, Survivin and B-Cell Maturation Antigen mRNA for Vaccination of Multiple Myeloma Patients.” Cancer Immunol Immunother 62 (8): 1381-92. doi:10.1007/s00262-013-1438-2.
Hoffmann, Peter R, Maddalena Panigada, Elisa Soprana, Frances Terry, Ivo Sah Bandar, Andrea Napolitano, Aaron H Rose, et al. 2015. “Preclinical Development of HIvax: Human Survivin Highly Immunogenic Vaccines.” Human Vaccines & Immunotherapeutics 11 (7): 1585-95. doi:10.1080/21645515.2015.1050572.
Johannsen, A, R Genolet, D F Legler, S A Luther, and I F Luescher. 2010. “Definition of Key Variables for the Induction of Optimal NY-ESO-1-Specific T Cells in HLA Transgene Mice.” J Immunol 185 (6): 3445-55. doi:10.4049/jimmunol.1001397.
Karanikas, V, S Khalil, T Kerenidi, K I Gourgoulianis, and A E Germenis. 2009. “Anti-Survivin Antibody Responses in Lung Cancer.” Cancer Lett 282 (2): 159-66. doi:10.1016/j.canlet.2009.03.015.
Karanikas, V, F Soukou, F Kalala, T Kerenidi, E S Grammoustianou, K I Gourgoulianis, and A E Germenis. 2008. “Baseline Levels of CD8+ T Cells against Survivin and Survivin-2B in the Blood of Lung Cancer Patients and Cancer-Free Individuals.” Clin Immunol 129 (2): 230-40. doi:10.1016/j.clim.2008.07.024.
Karbach, J, A Neumann, K Brand, C Wahle, E Siegel, M Maeurer, E Ritter, et al. 2012. “Phase I Clinical Trial of Mixed Bacterial Vaccine (Coley's Toxins) in Patients with NY-ESO-1 Expressing Cancers: Immunological Effects and Clinical Activity.” Clin Cancer Res 18 (19): 5449-59. doi:10.1158/1078-0432.CCR-12-1116.
Kenter, G G, M J Welters, A R Valentijn, M J Lowik, D M Berends-van der Meer, A P Vloon, F Essahsah, et al. 2009. “Vaccination against HPV-16 Oncoproteins for Vulvar Intraepithelial Neoplasia.” N Engl J Med 361 (19): 1838-47. doi:10.1056/NEJMoa0810097.
Khan, Salma, Heather Ferguson Bennit, Malyn May Asuncion Valenzuela, David Turay, Carlos J Diaz Osterman, Ron B Moyron, Grace E Esebanmen, Arjun Ashok, and Nathan R Wall. 2015. “Localization and Upregulation of Survivin in Cancer Health Disparities: A Clinical Perspective.” Biologics: Targets & Therapy 9 (January): 57-67. doi:10.2147/BTT.S83864.
Kim, E K, H I Cho, S H Yoon, M J Park, H J Sohn, H J Kim, S T Oh, and T G Kim. 2008. “Efficient Generation of Survivin-Specific Cytotoxic T Lymphocytes from Healthy Persons in Vitro: Quantitative and Qualitative Effects of CD4+ T Cells.” Vaccine 26 (32): 3987-97. doi: 10.1016/j.vaccine.2008.05.036.
Kim, H J, and Harvey Cantor. 2014. “CD4 T-Cell Subsets and Tumor Immunity: The Helpful and the Not-so-Helpful.” Cancer Immunology Research 2 (2): 91-98. doi:10.1158/2326-6066.CIR-13-0216.
Krieg, A M, A K Yi, S Matson, T J Waldschmidt, G A Bishop, R Teasdale, G A Koretzky, and D M Klinman. 1995. “CpG Motifs in Bacterial DNA Trigger Direct B-Cell Activation.” Nature 374 (6522): 546-49. doi:10.1038/374546a0.
Lennerz, V, S Gross, E Gallerani, C Sessa, N Mach, S Boehm, D Hess, et al. 2014. “Immunologic Response to the Survivin-Derived Multi-Epitope Vaccine EMD640744 in Patients with Advanced Solid Tumors.” Cancer Immunol Immunother 63 (4): 381-94. doi:10.1007/s00262-013-1516-5.
Leroux-Roels, Geert. 2010. “Unmet Needs in Modern Vaccinology: Adjuvants to Improve the Immune Response.” Vaccine 28 Suppl 3 (August): C25-36. doi:10.1016/j.vaccine.2010.07.021.
Li, F. 2005. “Role of Survivin and Its Splice Variants in Tumorigenesis.” British Journal of Cancer 92 (2): 212-16. doi:10.1038/sj.bjc.6602340.
Li, F, G Ambrosini, E Y Chu, J Plescia, S Tognin, P C Marchisio, and D C Altieri. 1998. “Control of Apoptosis and Mitotic Spindle Checkpoint by Survivin.” Nature 396 (6711): 580-84. doi:10.1038/25141.
Lingnau, K, K Riedl, and A von Gabain. 2007. “IC31 and IC30, Novel Types of Vaccine Adjuvant Based on Peptide Delivery Systems.” Expert Rev Vaccines 6 (5): 741-46. doi:10.1586/14760584.6.5.741.
Lladser, A, M Parraga, L Quevedo, M Carmen Molina, S Silva, A Ferreira, R Billetta, and G Quest A F. 2006. “Naked DNA Immunization as an Approach to Target the Generic Tumor Antigen Survivin Induces Humoral and Cellular Immune Responses in Mice.” Immunobiology 211 (1-2): 11-27. doi: 10.1016/j.imbio.2005.08.002.

Lladser, Alvaro, Karl Ljungberg, Helena Tufvesson, Marcella Tazzari, Anna Karin Roos, Andrew F G Quest, and Rolf Kiessling. 2010. “Intradermal DNA Electroporation Induces Survivin-Specific CTLs, Suppresses Angiogenesis and Confers Protection against Mouse Melanoma.” Cancer Immunology, Immunotherapy 59 (1): 81-92. doi:10.1007/s00262-009-0725-4.
Luckow, Verne A., and Max D. Summers. 1988. “Trends in the Development of Baculovirus Expression Vectors.” Bio/Technology 6 (1). Nature Publishing Company: 47-55. doi: 10.1038/nbt0188-47.
Megliorino, R, F D Shi, X X Peng, X Wang, E K Chan, E M Tan, and J Y Zhang. 2005. “Autoimmune Response to Anti-Apoptotic Protein Survivin and Its Association with Antibodies to p53 and c-Myc in Cancer Detection.” Cancer Detect Prev 29 (3): 241-48. doi:10.1016/j.cdp.2005.03.002.
Merrifield, R. B. 1963. “Solid Phase Peptide Synthesis. I. The Synthesis of a Tetrapeptide.” Journal of the American Chemical Society 85 (14). American Chemical Society: 2149-54. doi:10.1021/ja00897a025.
Miconnet, I, S Koenig, D Speiser, A Krieg, P Guillaume, J C Cerottini, and P Romero. 2002. “CpG Are Efficient Adjuvants for Specific CTL Induction against Tumor Antigen-Derived Peptide.” J Immunol 168 (3): 1212-18. http://www.ncbi.nlm.nih.gov/pubmed/11801657.
Miconnet, Isabelle, Sylvain Koenig, Daniel Speiser, Philippe Guillaume, Jean-charles Cerottini, and Pedro Romero. 2013. “CpG Are Efficient Adjuvants for Specific CTL Induction Against Tumor Antigen-Derived Peptide.”
Minoda, Masaya, Teruya Kawamoto, Takeshi Ueha, Etsuko Kamata, Masayuki Morishita, Risa Harada, Mitsunori Toda, et al. 2015. “Antitumor Effect of YM155, a Novel Small-Molecule Survivin Suppressant, via Mitochondrial Apoptosis in Human MFH/UPS.” International Journal of Oncology, July.doi:10.3892/ijo.2015.3077.
Nagaraj, S, V Pisarev, L Kinarsky, S Sherman, C Muro-Cacho, D C Altieri, and D I Gabrilovich. 2007. “Dendritic Cell-Based Full-Length Survivin Vaccine in Treatment of Experimental Tumors.” J Immunother 30 (2): 169-79. doi:10.1097/01.cji.0000211329.83890.ba.
Necochea-Campion, Rosalia de, Chien-Shing Chen, Saied Mirshahidi, Frank D Howard, and Nathan R Wall. 2013. “Clinico-Pathologic Relevance of Survivin Splice Variant Expression in Cancer.” Cancer Letters 339 (2): 167-74. doi:10.1016/j.canlet.2013.06.007.
Olie, R A, A P Simoes-Wust, B Baumann, S H Leech, D Fabbro, R A Stahel, and U Zangemeister-Wittke. 2000. “A Novel Antisense Oligonucleotide Targeting Survivin Expression Induces Apoptosis and Sensitizes Lung Cancer Cells to Chemotherapy.” Cancer Res 60 (11): 2805-9. http://www.ncbi.nlm.nih.gov/pubmed/10850418.
Ostrand-Rosenberg, S. 2005. “CD4+T Lymphocytes: A Critical Component of Antitumor Immunity.” Cancer Invest 23 (5): 413-19. http://www.ncbi.nlm.nih.gov/pubmed/16193641.
Otto, K, M H Andersen, A Eggert, P Keikavoussi, L O Pedersen, J C Rath, M Bock, et al. 2005. “Lack of Toxicity of Therapy-Induced T Cell Responses against the Universal Tumour Antigen Survivin.” Vaccine 23 (7): 884-89. doi:10.1016/j.vaccine.2004.08.007.
Pajot, A, M L Michel, N Fazilleau, V Pancre, C Auriault, D M Ojcius, F A Lemonnier, and Y C Lone. 2004. “A Mouse Model of Human Adaptive Immune Functions: HLA-A2.1-/HLA-DR1-Transgenic H-2 Class I-/class II-Knockout Mice.” Eur J Immunol 34 (11): 3060-69. doi:10.1002/eji.200425463.
Pérez, O, M Lastre, O Cabrera, J del Campo, G Bracho, M Cuello, J Balboa, et al. “New Vaccines Require Potent Adjuvants like AFPL1 and AFCol.” Scandinavian Journal of Immunology 66 (2-3): 271-77. doi:10.1111/j.1365-3083.2007.01981.x.
Piesche, M, Y Hildebrandt, F Zettl, B Chapuy, M Schmitz, G Wulf, L Trumper, and R Schroers. 2007. “Identification of a Promiscuous HLA DR-Restricted T-Cell Epitope Derived from the Inhibitor of Apoptosis Protein Survivin.” Hum Immunol 68 (7): 572-76. doi:10.1016/j.humimm.2007.03.007.
Pouwels, P. H., B. E. Enger-Valk, and W. J. Brammar. 1985. Cloning Vectors: A Laboratory Manual, Partie 1. Vol. 1985. https://books.google.fr/books/about/Cloningvectors.html?id=IWXwAAAAMAAJ&pgis=1.
Quakkelaar, Esther D., and Cornelis J M Melief. 2012. Experience with Synthetic Vaccines for Cancer and Persistent Virus Infections in Nonhuman Primates and Patients. Advances in Immunology. 1st ed. Vol. 114. Elsevier Inc. doi:10.1016/B978-0-12-396548-6.00004-4.
Rapoport, Aaron P, Nicole A Aqui, Edward A Stadtmauer, Dan T Vogl, Yin Yan Xu, Michael Kalos, Ling Cai, et al. 2014. “Combination Immunotherapy after ASCT for Multiple Myeloma Using MAGE-A3/Poly-ICLC Immunizations Followed by Adoptive Transfer of Vaccine-Primed and Costimulated Autologous T Cells.” Clinical Cancer Research: An Official Journal of the American Association for Cancer Research 20 (5). American Association for Cancer Research: 1355-65. doi:10.1158/1078-0432.CCR-13-2817.
Reker, S, A Meier, L Holten-Andersen, I M Svane, J C Becker, P thor Straten, and M H Andersen. 2004. “Identification of Novel Survivin-Derived CTL Epitopes.” Cancer Biol Ther 3 (2): 173-79. http://www.ncbi.nlm.nih.gov/pubmed/14726703.
Reker, Sine, Jürgen C Becker, Inge Marie Svane, Elisabeth Ralfkiaer, Per-thor Straten, and Mads Hald Andersen. 2004. “HLA-B35-Restricted Immune Responses against Survivin in Cancer Patients.” International Journal of Cancer. Journal International Du Cancer 108 (6): 937-41. doi:10.1002/ijc.11634.
Rittig, Susanne M, Maik Haentschel, Katrin J Weimer, Annkristin Heine, Martin R Muller, Wolfram Brugger, Marius S Horger, et al. 2011. “Intradermal Vaccinations with RNA Coding for TAA Generate CD8+ and CD4+ Immune Responses and Induce Clinical Benefit in Vaccinated Patients.” Molecular Therapy: The ournal of the American Society of Gene Therapy 19 (5). Nature Publishing Group: 990-99. doi:10.1038/mt.2010.289.
Rohayem, J, P Diestelkoetter, B Weigle, A Oehmichen, M Schmitz, J Mehlhorn, K Conrad, and E P Rieber. 2000. “Antibody Response to the Tumor-Associated Inhibitor of Apoptosis Protein Survivin in Cancer Patients.” Cancer Res 60 (7): 1815-17. http://www.ncbi.nlm.nih.gov/pubmed/10766164.
Rosalia, R A, E D Quakkelaar, A Redeker, S Khan, M Camps, J W Drijfhout, A L Silva, et al. 2013. “Dendritic Cells Process Synthetic Long Peptides Better than Whole Protein, Improving Antigen Presentation and T-Cell Activation.” Eur J Immunol 43 (10): 2554-65. doi:10.1002/eji.201343324.
Schaft, Niels, Jan Dörrie, Peter Thumann, Verena E Beck, Ina Müller, Erwin S Schultz, Eckhart Kimpgen, Detlef Dieckmann, and Gerold Schuler. 2005. “Generation of an Optimized Polyvalent Monocyte-Derived Dendritic Cell Vaccine by Transfecting Defined RNAs after rather than before Maturation.” Journal of Immunology (Baltimore, Md.: 1950) 174 (5): 3087-97. http://www.ncbi.nlm.nih.gov/pubmed/15728524.
Schaue, D, B Comin-Anduix, A Ribas, L Zhang, L Goodglick, J W Sayre, A Debucquoy, K Haustermans, and W H McBride. 2008. “T-Cell Responses to Survivin in Cancer Patients Undergoing Radiation Therapy.” Clin Cancer Res 14 (15): 4883-90. doi:10.1158/1078-0432.CCR-07-4462.
Schmitz, M, P Diestelkoetter, B Weigle, F Schmachtenberg, S Stevanovic, D Ockert, H G Rammensee, and E P Rieber. 2000. “Generation of Survivin-Specific CD8+T Effector Cells by Dendritic Cells Pulsed with Protein or Selected Peptides.” Cancer Res 60 (17): 4845-49. http://www.ncbi.nlm.nih.gov/pubmed/10987296.
Sharma, Rajesh K., Esma S. Yolcu, Abhishek K. Srivastava, and Haval Shirwan. 2013. “CD4+ T Cells Play a Critical Role in the Generation of Primary and Memory Antitumor Immune Responses Elicited by SA-4-1BBL and TAA-Based Vaccines in Mouse Tumor Models.” PLoS ONE 8 (9): 1-8. doi:10.1371/journal.pone.0073145.
Shedlock, Devon J, and Hao Shen. 2003. “Requirement for CD4 T Cell Help in Generating Functional CD8 T Cell Memory.” Science (New York, N.Y.) 300 (5617): 337-39. doi:10.1126/science.1082305.
Siegel, S, J Steinmann, N Schmitz, R Stuhlmann, P Dreger, and M Zeis. 2004. “Identification of a Survivin-Derived Peptide That Induces HLA-A*0201-Restricted Antileukemia Cytotoxic T Lymphocytes.” Leukemia 18 (12): 2046-47. doi:10.1038/sj.leu.2403510.
Siegel, S, A Wagner, N Schmitz, and M Zeis. 2003. “Induction of Antitumour Immunity Using Survivin Peptide-Pulsed Dendritic Cells in a Murine Lymphoma Model.” Br J Haematol 122 (6): 911-14. http://www.ncbi.nlm.nih.gov/pubmed/12956760.
Speiser, D E, D Lienard, N Rufer, V Rubio-Godoy, D Rimoldi, F Lejeune, A M Krieg, J C Cerottini, and P Romero. 2005. “Rapid and Strong Human CD8+ T Cell Responses to Vaccination with Peptide, IFA, and CpG Oligodeoxynucleotide 7909.” J Clin Invest 115 (3): 739-46. doi:10.1172/JCI23373.
Speiser, D E, and P Romero. 2010. “Molecularly Defined Vaccines for Cancer Immunotherapy, and Protective T Cell Immunity.” Semin Immunol 22 (3): 144-54. doi:10.1016/j.smim.2010.03.004.
Steinhagen, Folkert, Takeshi Kinjo, Christian Bode, and Dennis M Klinman. 2011. “TLR-Based Immune Adjuvants.” Vaccine 29 (17): 3341-55. doi:10.1016/j.vaccine.2010.08.002.
Tanaka, Makito, Marcus O Butler, Sascha Ansén, Osamu Imataki, Alla Berezovskaya, Lee M Nadler, and Naoto Hirano. 2011. “Induction of HLA-DP4-Restricted Anti-Survivin Th1 and Th2 Responses Using an Artificial Antigen-Presenting Cell.” Clinical Cancer Research: An Official Journal of the American Association for Cancer Research 17 (16): 5392-5401. doi:10.1158/1078-0432.CCR-10-3083.
Tokunaga, T, H Yamamoto, S Shimada, H Abe, T Fukuda, Y Fujisawa, Y Furutani, O Yano, T Kataoka, and T Sudo. 1984. “Antitumor Activity of Deoxyribonucleic Acid Fraction from Mycobacterium Bovis BCG. I. Isolation, Physicochemical Characterization, and Antitumor Activity.” Journal of the National Cancer Institute 72 (4): 955-62. http://www.ncbi.nlm.nih.gov/pubmed/6200641.
Trepiakas, Redas, Annika Berntsen, Sine Reker Hadrup, Jon Bjorn, Poul F Geertsen, Per Thor Straten, Mads H Andersen, et al. 2010. “Vaccination with Autologous Dendritic Cells Pulsed with Multiple Tumor Antigens for Treatment of Patients with Malignant Melanoma: Results from a Phase I/II Trial.” Cytotherapy 12 (6): 721-34. doi:10.3109/14653241003774045.
Turksma, Annelies W, Hetty J Bontkes, Janneke J Ruizendaal, Kirsten B J Scholten, Johanneke Akershoek, Shakila Rampersad, Laura M Moesbergen, et al. 2013. “Exploring Dendritic Cell Based Vaccines Targeting Survivin for the Treatment of Head and Neck Cancer Patients.” Journal of Translational Medicine 11: 152. doi:10.1186/1479-5876-11-152.
Valmori, D, N E Souleimanian, V Tosello, N Bhardwaj, S Adams, D O'Neill, A Pavlick, et al. 2007. “Vaccination with NY-ESO-1 Protein and CpG in Montanide Induces Integrated antibody/Th1 Responses and CD8 T Cells through Cross-Priming.” Proc Natl Acad Sci USA 104 (21): 8947-52. doi:0703395104 [pii]10.1073/pnas.0703395104.
van Poelgeest, Mariette I E, Marij J P Welters, Edith M G van Esch, Linda F M Stynenbosch, Gijs Kerpershoek, Els L van Persijn van Meerten, Muriel van den Hende, et al. 2013. “HPV16 Synthetic Long Peptide (HPV16-SLP) Vaccination Therapy of Patients with Advanced or Recurrent HPV16-Induced Gynecological Carcinoma, a Phase II Trial.” J Transl Med 11 (January): 88. doi:10.1186/1479-5876-11-88.
Vermeij, R, N Leffers, B N Hoogeboom, I L Hamming, R Wolf, A K Reyners, B H Molmans, et al. 2012. “Potentiation of a p53-SLP Vaccine by Cyclophosphamide in Ovarian Cancer: A Single-Arm Phase II Study.” Int J Cancer 131 (5): E670-80. doi:10.1002/ijc.27388.

Waligórska-Stachura, Joanna, Anna Jankowska, Ryszard Wazko, Wlodzimierz Liebert, Maciej Biczysko, Agata Czarnywojtek, Daria Baszko-Blaszyk, Violet Shimek, and Marek Ruchala. 2012. “Survivin—Prognostic Tumor Biomarker in Human Neoplasms—Review.” Ginekologia Polska 83 (7): 537-40. http://www.ncbi.nlm.nih.gov/pubmed/22880480.
Wang, X F, J Kerzerho, O Adotevi, H Nuyttens, C Badoual, G Munier, S Oudard, S Tu, E Tartour, and B Maillere. 2008. “Comprehensive Analysis of HLA-DR- and HLA-DP4-Restricted CD4+ T Cell Response Specific for the Tumor-Shared Antigen Survivin in Healthy Donors and Cancer Patients.” J Immunol 181 (1): 431-39. http://www.ncbi.nlm.nih.gov/pubmed/18566409.
Wick, D A, S D Martin, B H Nelson, and J R Webb. 2011. “Profound CD8+ T Cell Immunity Elicited by Sequential Daily Immunization with Exogenous Antigen plus the TLR3 Agonist poly(I:C).” Vaccine 29 (5): 984-93. doi:S0264-410X(10)01670-1 [pii]10.1016/j.vaccine.2010.11.036.
Widenmeyer, M, H Griesemann, S Stevanovic, S Feyerabend, R Klein, S Attig, J Hennenlotter, et al. 2012. “Promiscuous Survivin Peptide Induces Robust CD4+ T-Cell Responses in the Majority of Vaccinated Cancer Patients.” Int J Cancer 131 (1): 140-49. doi:10.1002/ijc.26365.
Wobser, M, P Keikavoussi, V Kunzmann, M Weininger, M H Andersen, and J C Becker. 2006. “Complete Remission of Liver Metastasis of Pancreatic Cancer under Vaccination with a HLA-A2 Restricted Peptide Derived from the Universal Tumor Antigen Survivin.” Cancer Immunol Immunother 55 (10): 1294-98. doi:10.1007/s00262-005-0102-x.
Xie, Hao, Wei Jiang, Shu-Yuan Xiao, and Xiuli Liu. 2013. “High Expression of Survivin Is Prognostic of Shorter Survival but Not Predictive of Adjuvant Gemcitabine Benefit in Patients with Resected Pancreatic Adenocarcinoma.” The Journal of Histochemistry and Cytochemistry: Official Journal of the Histochemistry Society 61 (2): 148-55. doi:10.1369/0022155412468137.
Yamamoto, S, T Yamamoto, T Kataoka, E Kuramoto, O Yano, and T Tokunaga. 1992. “Unique Palindromic Sequences in Synthetic Oligonucleotides Are Required to Induce IFN [correction of INF] and Augment IFN-Mediated [correction of INF] Natural Killer Activity.” Journal of Immunology (Baltimore, Md.: 1950) 148 (12): 4072-76. http://www.ncbi.nlm.nih.gov/pubmed/1376349.
Yang, Dun, Alana Welm, and J Michael Bishop. 2004. “Cell Division and Cell Survival in the Absence of Survivin.” Proceedings of the National Academy of Sciences of the United States of America 101 (42): 15100-105. doi:10.1073/pnas.0406665101.
Yang, Zhao, Li Wang, Huiming Wang, Xiaoyun Shang, Wei Niu, Jintao Li, and YuZhang Wu. 2008. “A Novel Mimovirus Vaccine Containing Survivin Epitope with Adjuvant IL-15 Induces Long-Lasting Cellular Immunity and High Antitumor Efficiency.” Molecular Immunology 45 (6): 1674-81. doi:10.1016/j.molimm.2007.10.026.
Zeestraten, E C, F M Speetjens, M J Welters, S Saadatmand, L F Stynenbosch, R Jongen, E Kapiteijn, et al. 2013. “Addition of Interferon-Alpha to the p53-SLP(R) Vaccine Results in Increased Production of Interferon-Gamma in Vaccinated Colorectal Cancer Patients: A Phase I/II Clinical Trial.” Int J Cancer 132 (7): 1581-91. doi:10.1002/ijc.27819.
Zeis, M, S Siegel, A Wagner, M Schmitz, M Marget, R Kuhl-Burmeister, I Adamzik, et al. 2003. “Generation of Cytotoxic Responses in Mice and Human Individuals against Hematological Malignancies Using Survivin-RNA-Transfected Dendritic Cells.” J Immunol 170 (11): 5391-97. http://www.ncbi.nlm.nih.gov/pubmed/12759413.
Zhang, Lei, Elaine F Lizzio, Trina Chen, and Steven Kozlowski. 2005. “Peptide Immunization Excludes Antigen-Specific T Cells from Splenic Lymphoid Compartments.” European Journal of Immunology 35 (3): 776-85. doi:10.1002/eji.200425479.
Zhang, Min, Alex Ho, Elizabeth H Hammond, Yoshiyuki Suzuki, R Scott Bermudez, R Jeffrey Lee, Michael Pilepich, et al. 2009. “Prognostic Value of Survivin in Locally Advanced Prostate Cancer: Study Based on RTOG 8610.” International ournal of Radiation Oncology, Biology, Physics 73 (4): 1033-42. doi:10.1016/j.ijrobp.2008.06.1489.
Zhao, Q, J Temsamani, P L Iadarola, Z Jiang, and S Agrawal. 1996. “Effect of Different Chemically Modified Oligodeoxynucleotides on Immune Stimulation.” Biochemical Pharmacology 51 (2): 173-82. http://www.ncbi.nlm.nih.gov/pubmed/8615886.
Zhou, F, M L Wang, H H Albert, P H Moore, and Y J Zhu. 2006. “Efficient Transient Expression of Human GM-CSF Protein in Nicotiana Benthamiana Using Potato Virus X Vector.” Appl Microbiol Biotechnol 72 (4): 756-62. doi:10.1007/s00253-005-0305-2.
Zhu, K, H Qin, S C Cha, S S Neelapu, W Overwijk, G A Lizee, J L Abbruzzese, et al. 2007. “Survivin DNA Vaccine Generated Specific Antitumor Effects in Pancreatic Carcinoma and Lymphoma Mouse Models.” Vaccine 25 (46): 7955-61. doi:10.1016/j.vaccine.2007.08.050.
Zwaveling, Sander, Sandra C Ferreira Mota, Jan Nouta, Mark Johnson, Grayson B Lipford, Rienk Offringa, Sjoerd H van der Burg, and Cornelis J M Melief. 2002. “Established Human Papillomavirus Type 16-Expressing Tumors Are Effectively Eradicated Following Vaccination with Long Peptides.” J Immunol 169 (1): 350-58. doi:10.4049/jimmunol.169.1.350.

1. An immunogenic composition comprising:
(a) at least one peptide derived from the alpha-isoform of survivin, or functional derivative thereof;
(b) at least one immunostimulatory adjuvant; and
(c) at least one adjuvant capable of creating a depot effect.

2. The immunogenic composition according to claim 1, wherein the at least one adjuvant capable of creating a depot effect in (c) is one or more adjuvant selected from the group consisting: alum, emulsion based formulations, mineral oil, non-mineral oil, and oil-in-water emulsion.

3. The immunogenic composition according to claim 1 or claim 2, wherein the at least one adjuvant capable of creating depot effect in (c) is a Montanide® adjuvant.

4. The immunogenic comprising according to claim 3, wherein the Montanide® adjuvant is one selected from the group consisting: MR-59, ASO3, ISA-51 VG and ISA-720 VG.

5. The immunogenic composition according to any preceding claim wherein at least one immunostimulatory adjuvant in (b) is an immunostimulatory oligonucleotide adjuvant comprising one or more unmethylated CpG motifs.

6. The immunogenic composition according to claim 5, wherein the immunostimulatory oligonucleotide adjuvant is an oligodeoxynucleotide-containing unmethylated CpG motif (CpG-ODN).

7. The immunogenic composition according to any preceding claim, wherein at least one immunostimulatory adjuvant in (b) comprises a granulocyte macrophage colony-stimulating factor (GM-CSF) adjuvant.

8. The immunogenic composition according to any preceding claim, wherein (b) comprises an unmethylated CpG motif and a granulocyte macrophage colony-stimulating factor (GM-CSF).

9. The immunogenic composition according to any preceding claim, wherein (b) comprises an oligodeoxynucleotide-containing unmethylated CpG motif (CpG-ODN) and a granulocyte macrophage colony-stimulating factor (GM-CSF).

10. The immunogenic composition according to any preceding claim, wherein (b) comprises an unmethylated CpG motif, and wherein (c) comprises a Montanide® adjuvant.

11. An immunogenic composition according to any preceding claim, wherein the at least one peptide in (a) comprises one or more selected from the group consisting:
(i) a peptide of 15 to 18 consecutive amino acids located between positions 17 to 34 (SEQ ID NO: 1) of the alpha-isoform of survivin;
(ii) a peptide of 15 to 27 consecutive amino acids located between positions 84 to 110 (SEQ ID NO: 2) of the alpha-isoform of survivin; or
(iii) a peptide of 15 to 21 consecutive amino acids located between positions 122 to 142 (SEQ ID NO: 3) of the alpha-isoform of survivin.

12. The immunogenic composition according to any preceding claim, wherein the at least one peptide in (a) comprises one or more selected from the group consisting:
(i) a peptide of 18 consecutive amino acids located between positions 17 to 34 (SEQ ID NO: 1) of the alpha-isoform of survivin;
(ii) a peptide of 27 consecutive amino acids located between positions 84 to 110 (SEQ ID NO: 2) of the alpha-isoform of survivin; or
(iii) a peptide of 21 consecutive amino acids located between positions 122 and 142 (SEQ ID NO: 3) of the alpha-isoform of survivin.

13. The immunogenic composition according to any preceding claim, wherein the at least one peptide in (a) comprises one or more selected from the group consisting:
(i) a peptide of 15 to 18 consecutive amino acids located between positions 17 to 34 (SEQ ID NO: 1) of the alpha-isoform of survivin, comprising at least the 15 consecutive amino acids between positions 20 to 34 (SEQ ID NO: 5), or positions 17 to 31 (SEQ ID NO: 4) of the alpha-isoform of survivin;
(ii) a peptide of 15 to 27 consecutive amino acids located between positions 84 to 110 (SEQ ID NO: 2) of the alpha-isoform of survivin, comprising at least the 15 consecutive amino acids between positions 84 to 98 (SEQ ID NO: 6), positions 90 to 104 (SEQ ID NO: 7), positions 93 to 107 (SEQ ID No: 8) or positions 96 to 110 (SEQ ID NO: 9) of the alpha-isoform of survivin; or
(iii) a peptide of 15 to 21 consecutive amino acids located between positions 122 to 142 (SEQ ID NO: 3) of the alpha-isoform of survivin, comprising at least the 15 consecutive amino acids between positions 122 to 136 (SEQ ID NO: 10) or 128 to 142 (SEQ ID NO: 11) of the alpha-isoform of survivin.

14. The immunogenic composition according to any preceding claim, wherein at least one peptide in (a) is labelled or complexed.

15. The immunogenic composition according to any preceding claim, wherein (a) comprises a polypeptide comprising a concatenation of at least two peptides, wherein at least one of said concatenated peptides is a peptide according to any of claims 11 to 14.

16. The immunogenic composition according to any preceding claim, wherein (a) comprises a lipopeptide, wherein the lipopeptide comprises a peptide according to any of claims 11 to 14 with a lipid added to an alpha-amino function or a reactive side chain of said peptide.

17. The immunogenic composition according to any preceding claim, wherein (a) comprises an expression vector, wherein the expression vector comprises a polynucleotide encoding a peptide, polypeptide or lipopeptide according to any of claims 11 to 16.

18. The immunogenic composition according to any preceding claim, comprising:
(a) (i) a peptide of 18 consecutive amino acids located between positions 17 to 34 (SEQ ID NO: 1) of the alpha-isoform of survivin;
(ii) a peptide of 27 consecutive amino acids located between positions 84 to 110 (SEQ ID NO: 2) of the alpha-isoform of survivin; and
(iii) a peptide of 21 consecutive amino acids located between positions 122 and 142 (SEQ ID NO: 3) of the alpha-isoform of survivin;
(b) at least one immunostimulatory adjuvant; and
(c) at least one adjuvant capable of creating a depot effect.

19. The immunogenic composition according to claim 18, wherein (b) comprises an oligodeoxynucleotide-containing unmethylated CpG motif (CpG-ODN) and wherein (c) comprises a Montanide® adjuvant.

20. The immunogenic composition according to any preceding claim, wherein the composition is capable of inducing a T-cell mediated immune response against survivin.

21. The immunogenic composition according to claim 20, wherein the T-cell mediated immune response comprises inducing survivin-specific CD4⁺ and/or CD8⁺ T lymphocytes.

22. The immunogenic composition according to any preceding claim, for use in the treatment of cancer.

23. The immunogenic composition according to any preceding claim, for use in the prophylactic or therapeutic immunization of a subject who has or may develop cancer.

24. The immunogenic composition according to any preceding claim, for use in the diagnosis, prognosis or therapeutic monitoring of cancer in a subject.

25. The immunogenic composition for use according to any of claims 22 to 24, wherein the cancer over-expresses survivin.

26. A kit of parts comprising:
(a) at least one peptide derived from the alpha-isoform of survivin, or functional derivative thereof;
(b) at least one adjuvant; and
instructions for preparing of an immunogenic composition according to any of claims 1 to 21.

27. A method of preparing an immunogenic composition according to any of claims 1 to 21.

28. A method of treating cancer, the method comprising administering the immunogenic composition according to any of claims 1 to 21 to a subject in need.

29. A method of prophylactic or therapeutic immunization of a subject who has or may develop cancer, the method comprising administering the immunogenic composition according to any of claims 1 to 21.

30. A method of diagnosis, prognosis or therapeutic monitoring of cancer in a subject, the method comprising administering the immunogenic composition according to any of claims 1 to 21.

31. The method according to any of claims 27 to 30, wherein the cancer over-expresses survivin.
